(19) United States
(12) Patent Application Publication
(10) Pub. No.: US 2019/0187155 A1
(43) Pub. Date: Jun. 20, 2019

(54) COMPOSITION AND METHODS FOR MEASURING ION CHANNEL ACTIVITY IN A CELL

(71) Applicant: LIFE TECHNOLOGIES CORPORATION, Carlsbad, CA (US)

(72) Inventors: Daniel BEACHAM, Eugene, OR (US); Kyle GEE, Springfield, OR (US); Aleksey RUKAVISHNIKOV, Eugene, OR (US)

(21) Appl. No.: 16/324,769

(22) PCT Filed: Aug. 16, 2017
(86) PCT No.: PCT/US2017/047152

§ 371 Date: Feb. 11, 2019

Related U.S. Application Data
(60) Provisional application No. 62/376,232, filed on Aug. 17, 2016.

(51) Int. Cl. G01N 33/68; C07D 493/10; G01N 33/84

(52) U.S. Cl. CPC G01N 33/6872; G01N 2333/705; G01N 33/84; C07D 493/10

(57) ABSTRACT

Compositions and methods for detecting the activity of an ion channel in a cell are described. The methods include providing a loading buffer solution to the cell, where the loading buffer includes a thallium ion indicator and optionally chloride ions, and providing a stimulus buffer that includes thallium ions to the cell. Providing the stimulus buffer can cause thallium ion influx into or efflux out of the cell through the ion channel. After providing the stimulus buffer, a change in at least one optical property of the thallium ion indicator is detected in response to thallium influx or efflux, thereby detecting the activity of the ion channel.

### TECHNICAL FIELD

Compositions, methods and kits for measuring the activity of an ion channel in a cell are described,

### BACKGROUND

Thallium ion influx can be used as a surrogate indicator of potassium ion channel activity in clonal cell lines loaded with the calcium ion indicator benzonthiazole calcium acetoxymethyl (BTC AM) ester or a thallium-sensitive fluorogenic dye. Current assays for monitoring potassium ion channels use thallium (I) ion, which selectively enters open potassium channels and binds to BTC, giving an optical readout of potassium ion channel activity. This method can be used to study the activation and/or inhibition of ion channels with drugs tested in high throughput screening (HTS) mode from a compound library. The methods based on binding to BTC, however, have significant drawbacks. Namely, when the assay is performed in the presence of chloride ions, thallium (I) ion forms thallium chloride (TlCl), which is poorly soluble and will precipitate out of solution at a concentration of about 4.5 mM or greater. Thus, the buffers used for current methods implementing BTC should be essentially free of chloride ion to prevent TlCl from precipitating out of solution to generate inconsistent data. The current methods, therefore, require the additional steps of washing and removal of buffers in which cells are normally grown in culture (e.g., chloride ion containing buffers). Moreover, because chloride is absent in these assays, the assays may be seen as not approximating physiological conditions.

As an alternative to BTC, HTS of potassium ion channel and transporter activities can be monitored using a fluorogenic dye that is sensitive to the presence of thallium ions. The fluorescent signal reported in this type of fluorescence-based assay can serve as a surrogate readout of the activity of a potassium ion channel or transporter that is permeant to thallium ions. Here, cells are loaded with non-fluorescent, thallium ion sensitive dye. Drugs to be screened are pre-incubated with the cells, and the microplates are loaded into the reader, where they are injected with a stimulus buffer containing a low level of thallium ions. The thallium ions freely flow through open potassium channels, acting as a surrogate for K⁺. When the potassium channel is stimulated, thallium flows into the cell and binds the fluorogenic dye, generating a fluorescent signal, proportional to channel activity in physiological saline conditions. However, an issue faced by existing fluorogenic dyes is that they are not sufficiently sensitive to detect very low levels of thallium in HTS assays (e.g., below about 100-500 μM). Thus, there is a need for improved, highly sensitive compounds that quantitatively detect thallium ions even at very low concentrations under physiologically-relevant conditions. In addition, existing fluorogenic compounds emit light that can often interfere with other fluorescent components in FITS assays, such as, e.g., green fluorescent proteins. Thus, there is a need for new fluorogenic compounds for use in HTS assays that emit light over a range of visible wavelengths in response to the presence of metal ions such a thallium when used in monitoring the activity of ion channels. There also is a need for a sensor outside of the FITC/green optical channel due to presence of green autofluorescence from compounds in drug libraries. Autofluorescence is known to occlude and/or confound measurements made in this channel. Thus, there is a need for a sensor that absorbs and emits light outside of the GFP/FITC channel and provides a meaningful counterscreen that works to both corroborate “hits” in a GFP/FITC screen, as well as discover potential therapeutics whose activity would otherwise be masked by intrinsic green chanel fluorescence.

### SUMMARY

In one aspect, a method for detecting the activity of a potassium ion channel in a cell is provided, including: a) contacting the cell with a loading buffer, wherein the cell comprises a potassium ion channel, wherein the loading buffer comprises a thallium ion indicator; b) applying a stimulus buffer to the cell, wherein the stimulus buffer comprises thallium ions, thereby causing thallium ion influx into the cell through the potassium ion channel; and c) measuring a change in at least one optical property of the thallium ion indicator in response to thallium influx, thereby detecting the activity of the potassium ion channel, wherein the thallium ion indicator has a structure represented as:

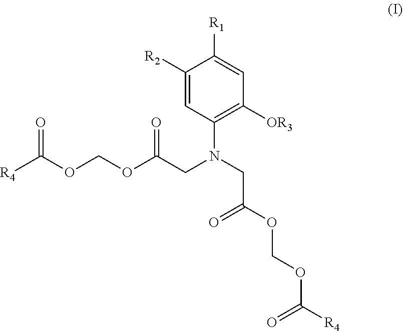

wherein R₂=H and R₁=

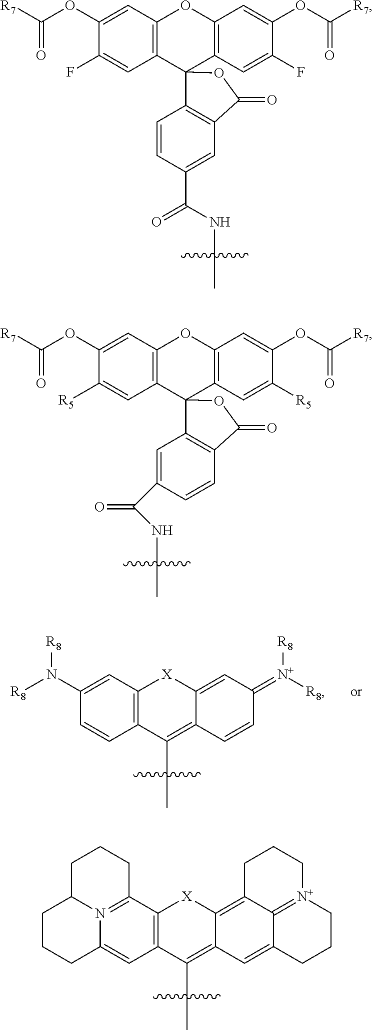

or wherein R₁=H and R₂=

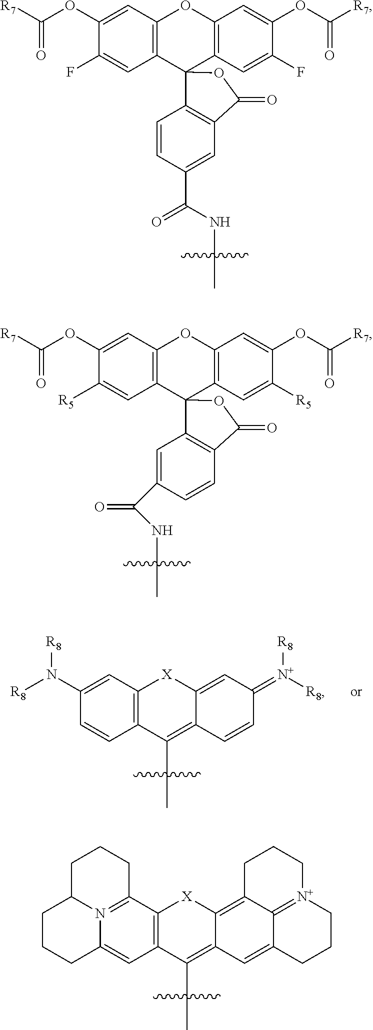

wherein X=O or (R₆)₂C; wherein R₃, R₄, R₆ and R₈ are independently C₁-C₆ alkyl; wherein R₅ is H or F; and wherein R₇ is H, CH₃ or C₂-C₆ alkyl, or a salt thereof.

In another aspect, a method for detecting the activity of a potassium ion channel in a cell is provided, including: a) contacting the cell with a loading buffer solution, wherein tree cell comprises a potassium ion channel, wherein the loading buffer solution comprises a thallium ion indicator, as disclosed herein, and a physiological concentration of chloride ions, applying a stimulus buffer to the cell, wherein the stimulus buffer comprises thallium ions, thereby causing thallium ion influx into the cell through the potassium ion channel; and measuring a change in at least one optical property of the thallium ion indicator in response to thallium influx, thereby detecting the activity of the potassium ion channel.

The stimulus buffer can include thallium ion concentrations of less than about 4.5 mM. The method can further include quantifying the levels of thallium ion influx, in the disclosed methods, at least one optical property of the thallium indicator (e.g., intensity, polarity, frequency, or optical density) can be assayed. The method can include measuring a change in the luminescence intensity of the thallium ion indica response to thallium ion influx. The loading buffer can be chloride free. The cell can be a mammalian cell. The method can further include washing the cells after applying the loading buffer to the cells. In some embodiments, the method does not involve washing the cells after the loading buffer is provided to the cells. The thallium can be in the form or a. salt. The thallium salt can be soluble in the loading buffer solution. For example, the thallium salt can be Tl₂SO₄, Tl₂CO₃, TlCl, TlOH, TlOAc, or TlNO₃. The method can further include adding a quencher to the loading buffer solution. The quencher can be substantially not cell permeant. For example, the quencher can be tartrazine, amaranth, acid red 37, congo red, trypan blue, brilliant black, or a combination thereof. In certain embodiments, the methods disclosed herein can further include adding an extracellular quencher to the loading buffer solution, whereby the emission of extracellular thallium ion indicator is quenched.

In another aspect, a kit is provided for detecting the activity of a potassium ion channel in a cell. The kit can include a loading buffer solution, wherein the loading buffer solution comprises chloride, a thallium ion indicator, and a stimulus buffer, wherein the stimulus buffer comprises thallium ion, and wherein the stimulus buffer causes thallium ion influx into the cell through the ion channel wherein the thallium ion indicator has a structure represented as:

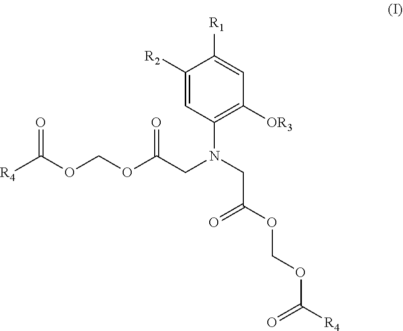

wherein R₇=H and R₁=

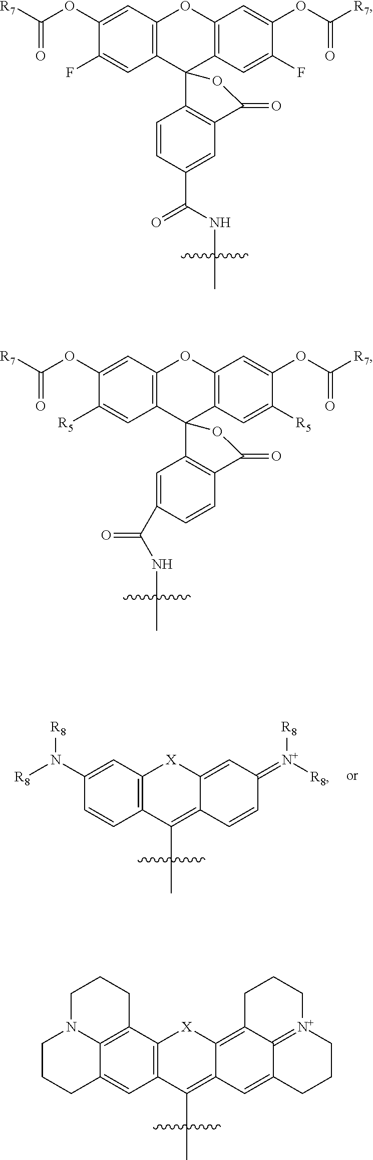

or wherein R₁=H and R₂=

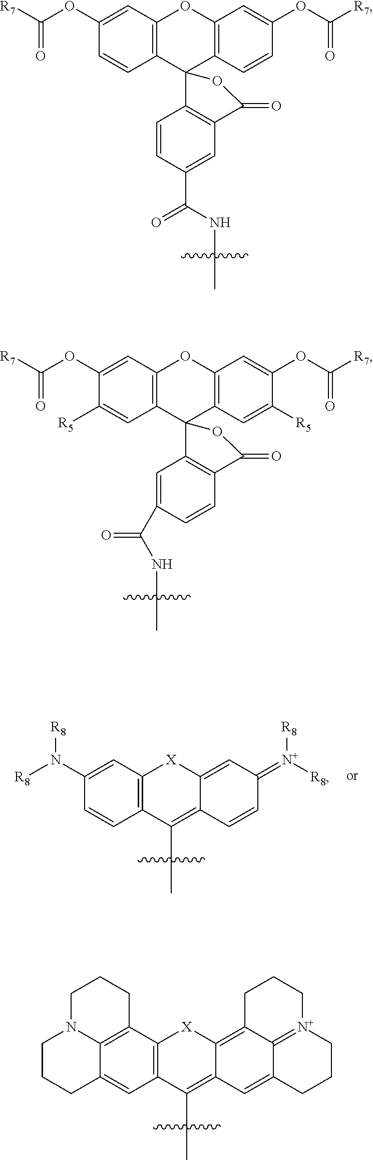

wherein X=O or (R₆)₂C; wherein R₃, R₄, R₆ and R₈ are independently C₁-C₆ alkyl; wherein R₅ is H or F; and wherein R₇ is H, CH₃ or C₂-C₆ alkyl, or a salt thereof.

In yet another aspect, a compound is provided having a structure represented as:

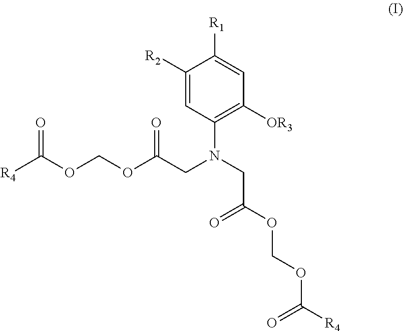

wherein R₂ =H and R₁ =

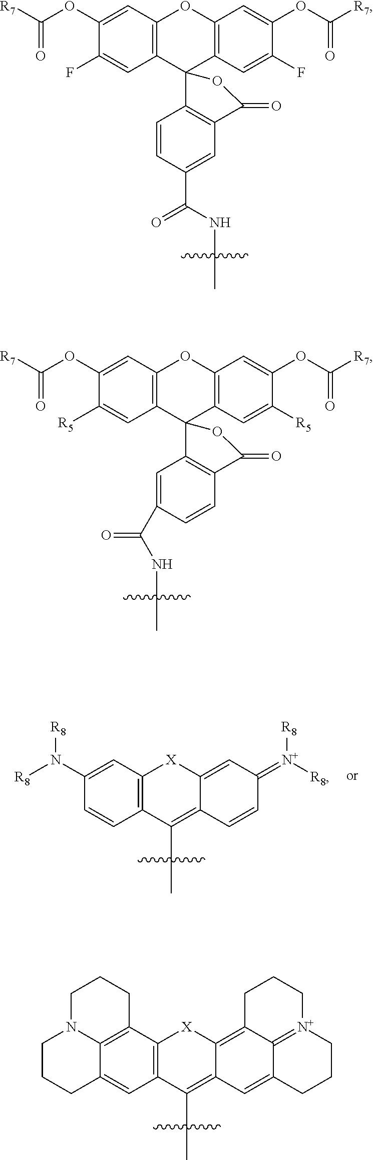

wherein X=O or (R₆)₂C; wherein R₃, R₄, R₆ and R₈ are independently C,- alkyl; wherein R₅ is H or F; and, wherein R₇ is H, CH₃ or C₂-C₆ alkyl, or a salt thereof, with the exception that if R₁ is

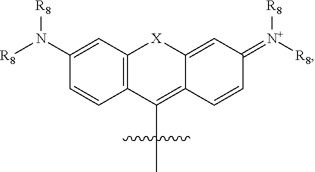

then each R₈ is not C₁ alkyl.

In yet another aspect, a compound is provided having a structure represented as:

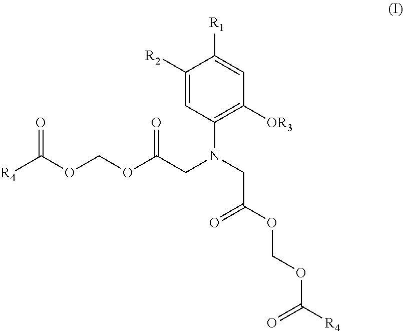

wherein R₁=H and R₂=

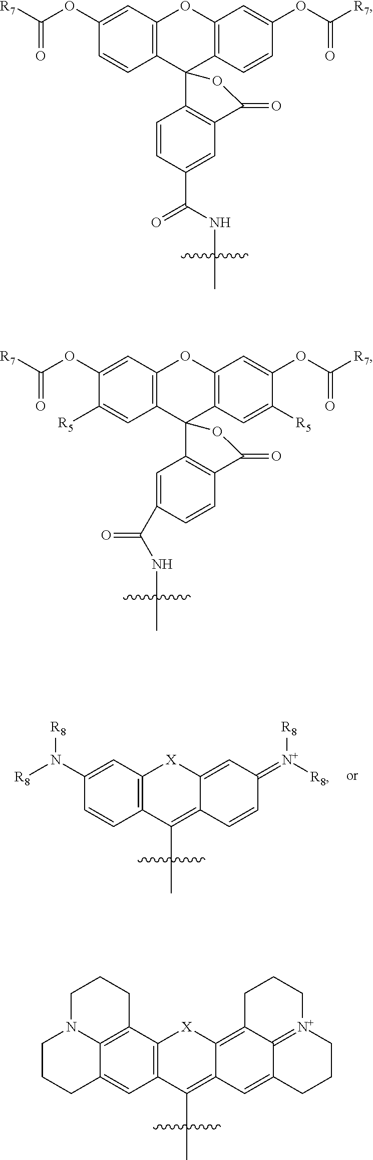

wherein X=O or (R₆)₂C; wherein R₃, R₄, R₆ and R₈ are independently C₁-C₆ alkyl; wherein R₅ is H or F; and, wherein R₇ is H, CH₃ or C₂-C₆ alkyl, or a salt thereof. In any of the compounds, kits, or methods disclosed herein at least one R₅, if present, can be F.

In yet another aspect, a fluorescent complex is provided including a compound as disclosed herein; and a thallium ion, wherein the complex emits light upon excitation at an appropriate spectral wavelength.

In yet another aspect, a composition is provided comprising a compound or complex as disclosed herein dissolved in an aqueous medium.

### BRIEF DESCRIPTION OF THE DRAWINGS

FIG. 1A shows a general chemical structure (I) and representative thallium ion indicators, wherein R₂ is H.

FIG. 1B shows representative thallium ion indicators, wherein R₁ is H in the chemical structure (1) showing in FIG. 1A.

FIG. 2 is the chemical structure for compound (1).

FIG. 3 is the chemical structure for compound (2).

FIG. 4 is the chemical structure for compound (3).

FIG. 5 is the chemical structure for compound (4).

FIG. 6 is the chemical structure for compound (5).

FIG. 7 is the chemical structure for compound (6).

FIG. 8 is the chemical structure for compound (7).

FIG. 9 is the chemical structure for compound (8).

FIG. 10 is a plot showing the evolution of fluorescence signal over time for cells loaded with Compound (9) or Compound (8) and tested in the thallium influx assay described herein. Fluorescence data from the samples are plotted over time as fold increase in signal (post stimulus) over baseline (pre stimulus). Signal amplitude is compared from an average of 5-10 individual wells loaded with the dye indicated. The larger response (signal amplitude) from compound 8 (upper traces) post stimulus indicates its superiority in the assay relative to compound (9) (lower traces).

FIG. 11 is the chemical structure of a representative thallium sensitive compound.

FIG. 12A and FIG. 12B together are a reaction scheme for the synthesis of bis(acetoxymethyl) 2,2′-((4-(3′,6′-diacetoxy-2′,7′-difluoro-3-oxo-3H-spiro[isobenofuran-1,9′-xanthene]-6-carboxamido)-2-methoxyphenyl)azanediyl)diacetate.

FIG. 13A and FIG. 13B together are a reaction scheme for the synthesis of bis(acetoxymethyl) 2,2′-(4-(3′,6′-diacetoxy-2′,7′-difluoro-3-oxo-3H-spiro[isobenzofuran-1,9′-xanthene]-5-carboxamido)-2-methoxyphenyl)azanediyl)diacetate.

FIG. 14 is a reaction scheme for the synthesis of bis(acetoxymethyl)-2,2′-((5-amino-2-methoxyphenyl)azanediyl)diacetate.

FIG. 15 is a reaction scheme for the synthesis of bis(acetoxymethyl) 2,2′-((5-(3′,6′-diacetoxy-2′,7′-difluoro-3-oxo-3H-spiro[isobenzofuran-1,9′-xanthene]-6-carboxamido)-2- methoxyphenyl)azanediyl)diacetate.

FIG. 16 is a reaction scheme for the synthesis of bis(acetoxymethyl) 2,2′-((5-(3′,6′-diacetoxy-2′,7′-difluoro-3-oxo-3H-spiro[isobenzofuran-1,9′-xanthene]-5- carboxamido)-2-methoxyphenyl)azanediyl)diacetate (Compound 9).

FIG. 17 is a reaction scheme for the synthesis of dimethyl-2,2′-((4-formyl-2-methoxyphenyl)azanediyl)diacetate and dimethyl-2,2′((5-formyl-2-methoxyphenyl)diacetate.

FIG. 18A and FIG. 18B together are a reaction scheme for the synthesis of N-(9-(3-(bis(2-(acetoxymethoxy)-2-oxoethyl)amino)-4-methoxyphenyl)-6-(dimethylamino)-3H-xahthen-3-ylidene)-N-methylmethanaminium bromide.

FIG. 19A and FIG. 19B together are a reaction scheme for the synthesis of N-(9-(4-(bis(2-(acetoxymethoxy)-2-oxoethyl)amino)-3-methoxyphenyl)-6-(dimethylamino)-3H-xahthen-3-ylidene)-N-methylmethanaminium bromide.

FIG. 20A and FIG. 20B together are a reaction scheme for the synthesis of N-(10-(4-(bis(2-(acetoxymethoxy)-2-oxoethyl)amino)-3-methoxyphenyl)-7-(dimethylamino)-9,9-dimethylanthracen-2(9H)-ylidene)-N-methytmethanaminium riftuoromethanesulfonate and N-(10-(3-(bis(2-(acetoxymethoxy)-2-oxoethyl)amino)-4-methoxyphenyl)-7-(dimethylamino)-9,9-dimethylanthracen-2(9H)-ylidene)-N-methylmethanaminium trifluoromethanesulfonate.

### DETAILED DESCRIPTION

Unless defined otherwise, all technical and scientific terms used herein have the same meaning as is commonly understood by one of ordinary skill in the art to which this invention belongs. All patents, applications, published applications and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, “a” or “an” means at least on or “one or more.”

As used herein, the term “about” when used to describe a numerical value, encompasses a range up to ±15% of that numerical value, unless the context clearly dictates otherwise.

While compositions and methods are described in terms of “comprising” various components or steps (interpreted as meaning “including, but not limited to”), the compositions and methods can also “consist essentially of” or “consist of” the various components and steps, such terminology should be interpreted as defining essentially closed-member groups.

Provided herein are compounds and compositions for use in methods for detecting the activity of an ion channel in a cell. As used herein, the term “cell” is intended to mean one or more cells. The cell can be in any environment, provided that the loading and stimulus buffers can be applied to the cell. In one embodiment, the cell is in an in vitro environment and the methods are performer using well-known cell culture techniques. In a more specific embodiment, the cell is in a cell culture suspension. In another specific embodiment, the cell is in a cell adhesion culture.

The disclosed methods can be practiced on any cell, provided the cell possesses or expresses an ion channel that is permeable to thallium ions. Examples of ion channels include, but are not limited to, potassium ion channels, ion channels that are linked to receptors, e.g., GIRK, and channel-linked receptors, e.g., GPCR, and ion transporters, e.g., glutamate transporters. The cells can normally possess or express the ion channels, or the ion channels can be introduced into he cells using well-known transfection and transformation techniques. Methods are provided for assaying cells expressing native levels of ion channel (e.g., non-engineered cells) and for assaying cells that have been modified (e.g., engineered) by the practitioner to include an ion channel.

The methods are not limited to a particular type of ion channel, provided that the channel is permeable to thallium ions. Thus, the types of ion channels that can be used in methods disclosed herein include, but are not limited to, ligand- or voltage-gated, stretch-activated cation channels, selective or non-selective cation channels.

Types of ligand-gated non-selective cation channels include, but are not limited to, acetylcholine receptors, glutamate receptors such as AMPA, kainate, and NMDA receptors, 5-hydroxytryptamine-gated receptor-channels, ATP-gated (P2X) receptor-channels, nicotinic acetylcholine-gated receptor-channels, vanilloid receptors, ryanodine receptor-channels, IP3 receptor-channels, cation channels activated in situ by intracellular cAMP, and cation channels activated in situ by intracellular cGMP.

Types of voltage-gated ion channels include, hut are not limned to, K⁺ and Na⁺ channels. The channels can be expressed exogenously or endogenously. The channels can he stably or transiently expressed in both native or in engineered cell lines.

In one aspect, methods are disclosed for detecting the activity of a potassium (K⁺) ion channel. Types of K⁺ channels include, but arc not limited to, KCNQI (KvLOTI), KCNQ2, KCNQ3, KCNQ4, KCNQ5, HERG, KCNEI (IeK, MinK). Kv1.5, Kir 3.1, Kir 3.2, Kir 3.3, Kir 3.4, Kir6.2, SUR2A, ROMKI, Kv2.1, Kv1.4, Kv9.9. Kir6, SUR2B, KCNQ2, KCNQ3, GIRK1, GIRK2, GIRK3, GIKK4, hlKl, KCNAl, SURl, Kv1.3, hERG. intracellular calcium-activated K⁺ channels, rat brain (BK2); mouse brain (BK1) and other types of K⁺ ion channels that are well-known to those skilled in the art.

The methods also can be used for detecting the activity of a sodium (Na⁺) ion channel. Types of Na⁺ channels include, but are not limited to rat brain I, II and III, human II and the like. Thallium flux-based assays, such as described herein, can be used, e.g., to study sodium channels using the NaV1.7 channel as a model target (sec. Du. Y., et al., ACS Chem Neurosci. 2015 Jun. 17; 6(6):871-8).

The methods described herein can also be applied to indirectly measure the activity of channel-linked receptors and signal transduction systems. Channel activity may be modulated from interactions between receptor subunits with ion channels, e.g., GPCR β-γ sub-units and GPCR-linked K⁺ channels, e.g., GIRKs, or by changes in the concentrations of messenger molecules such as calcium, lipid metabolites, or cyclic nucleotides, which modulate the ion channel activity.

Accordingly, the disclosed methods can be used for monitoring, detecting and/or measuring the activity of intracellular events that are known to cause changes in ion channel permeability. Intracellular activity can include, but is not limited to protein phosphorylation or de-phosphorylation, up-regulation or down-regulation of transcription, cellular division, cellular apoptosis, receptor dimerization, and the like. Thus, the measurement or detection of such intracellular events can also serve as an indirect detection or measure of the ion channels, if so desired.

In addition, G-coupled protein receptors also can be utilized in the described methods. Examples of G-coupled protein receptors include, but are not limited to, muscarinic acetylcholine receptors (mAChR), adrenergic receptors, serotonin receptors, dopamine receptors, angiotensin receptors, adenosine receptors, bradykinin receptors, metabotropic excitatory amino acid receptors and the like.

Another type of indirect assay involves determining the activity of receptors which, when activated, result in a change in the level of intracellular cyclic nucleotides, e.g., cAMP, cGMP. For example, activation of some dopamine, serotonin, metabotropic glutamate receptors and muscarinic acetylcholine receptors results in an increase or decrease in the cAMP or cGMP levels of the cytoplasm. Furthermore, some cyclic nucleotide-gated ion channels, e.g., rod photoreceptor cell channels and olfactory neuron channels, are known to be permeable to cations upon activation by binding of cAMP or cGMP. Thus, a change in cytoplasmic ion levels, caused by a change in the amount of cyclic nucleotide activation of photo-receptor or olfactory neuron channels, can be used to determine the function of receptors that cause a change in cAMP or cGMP levels when activated. In one embodiment, a reagent that increases or decreases intracellular nucleotide levels is added to the cell, e.g., forskolin, prior to the addition of a receptor-activating compound. For example, if activation of a receptor is known or suspected to result in a decrease in cyclic nucleotide levels, forskolin, which is known to increase intracellular levels of nucleotide levels, may be added to the cells prior to adding a receptor-activating compound to the cells in the assay.

Cells used for this type of assay can be generated by co-transfection of a host cell with DNA encoding an ion channel, such as hERG, and DNA encoding a channel-linked receptor which, when activated, cause a change in cyclic nucleotide levels in the cytoplasm.

Receptors, include, but are not limited to, muscarinic receptors, e.g., human M2, rat M3, human M4, human M5, and the like. Other receptors include, but are not limited to, neuronal nicotinic acetylcholine receptors, the human α₂, human α₃, and human β₂, human α₅, subtype rat α₂ subunit, rat α₃ subunit, rat α₄ subunit, rat α₅ subunit, chicken α₇ subunit, rat β2 subunit, rat β₃ subunit at β4 subunit, combinations of the rat α a subunits, rat NMDAR1 receptor, mouse NMDA e1 receptor, at NMDAR2A, NMDAR2B and NMDAR2C receptors, rat metabotropic mGluR1 receptor, rat metabotropic mGluR2, mGluR3 and mGluR4 receptors, rat metabotropic mGluR5 receptor and the like. Other receptors include, but are not limited to, adrenergic receptors, e.g., human beta 1, human alpha 2, hamster beta 2, and the like. Still other receptors include, but are not limited to, dopamine receptors, serotonin receptors and serotonin receptors, e.g., human D2, mammralian dopamine D2 receptor, rat dopamine receptor, human 5HT1a, serotonin 5HT1C. receptor, human 5HT1D, rat 5HT2, rat 5HT1c and the like.

The term “ion channel” also includes ion transporters. Examples of ion transporters include, but are not limited to, neurotransmitter ion transporters, e.g., dopamine ion transporter, glutamate ion transporter or serotonin ion transporter, sodium-potassium ATPase, proton-potassium ATPase, sodium/calcium exchanger, and potassium-chloride ion co-transporter.

Types of cells that can be used in the described methods include, but are not limited to, bacterial cells, yeast cells, plant cells and animal cells (e.g., insect cells, avian cells, and mammalian cells).

To perform the disclosed methods, a loading buffer is provided to the cells. The loading buffer can be a solution and can include an environmentally sensitive agent and optionally can include chloride ion. As used herein, an “environmentally sensitive agent” is a compound, such as a dye, where at least one optical property of the compound changes in response to one aspect of its immediate environment. In a particular embodiment, at least one optical property of the environmentally sensitive agent can be sensitive to thallium ions. For example, the environmentally sensitive agent can be a luminescent dye. In some embodiments, the environmentally sensitive agent is a fluorogenic dye that is sensitive to thallium ions. For example, the fluorogenic dye inside a cell can be relatively non-fluorescent in the absence of thallium ions but significantly more fluorescent in the presence of thallium ions in sufficient concentrations.

In addition, the loading buffer can include additional components, such as but not limited to, serum albumin, transferrin, L-glutamine, lipids, antibiotics, β-mercaptoethanol, vitamin minerals, ATP and similar components may be present. The loading buffer can also include at least one inhibitor of organic ion transport, such as, but not limited to, benzbromarone, probenecid allopurinol, colchicine and sulfinpyrazole. Examples of vitamins that may be present include, but are not limited to vitamins A, B₁, B₂, B₃, B₅, B₆, B₉, B₁₂, C, D₁, D₂, D₃, D₄, D₅, E, tocotrienols, K₁ and K₂. One of skill in the art can determine the optimal concentration of minerals, vitamins, ATP, lipids, essential fatty acids, etc., for use in a given culture. The concentration of supplements may, for example, be from about 0.001 μM to about 1 mM. or more. Specific examples of concentrations at which the supplements may be provided include, but are not limited to about 0.005 μM, 0.01 μM, 0.05 μM, 0.1 μM, 0.5 μM, 1.0 μM, 2.0 μM, 2.5 μM, 3.0 μM, 4.0 μM, 5.0 μM, 10 μM, 20 μM, or 100 μM.

In certain aspects, the environmentally sensitive, agent is a compound that is sensitive to the presence of thallium ions, in which case the compound can be referred interchangeably to a “thallium ion sensitive agent” or “thallium indicator.” Thallium ion sensitive agents can be employed as an indicator of the flux of thallium ion across the cell membrane and are sufficiently sensitive so as to produce detectable changes in at least one optical property in response to changes in the concentration of the thallium ions in the cell cytoplasm. Types of thallium ion sensitive agents that can produce a detectable include, but are not limited to, fluorescent compounds and non-fluorescent compounds. The thallium ion sensitive agents can be hydrophilic or hydrophobic. Suitable thallium sensitive agents for use in the assays disclosed herein can be screened using the Thallium Ion Sensitivity Assay described in Example 14.

In certain aspects, the thallium ion sensitive agent can be a fluorescent dye. Numerous examples of thallium ion sensitive fluorescent compounds that can be loaded into cells and are sensitive to thallium ions are described herein. In one specific embodiment, the compound is selected to detect low concentrations of thallium ions (e.g., 1 mM or less).

The thallium sensitive fluorescent compound can be loaded into the cell by contacting the cells with a loading buffer comprising the dye or a membrane-permeable derivative of the dye. Loading the cells with the dye can be further facilitated by using a more hydrophobic form of the dye. For certain applications, it can be desirable to provide a thallium indicator with a cleavable hydrophobic moiety. For example, thallium indicators with a cleavable hydrophobic moiety may readily enter the cell through the cell membrane. Once inside the cell, the moiety may be cleaved by an agent (e.g., enzyme) within the cell to produce a less hydrophobic compound, which remains trapped within the cell. The cleavable moiety may be any moiety susceptible to cleavage by an enzyme (e.g., esterases, lipases, phospholipases, and the like). Representative cleavable moieties include, for example, hydrophobic moieties, such as acetoxymethyl (AM) ester. In some embodiments, a thallium indicator can be a dye in the form of an acetoxymethyl ester (AM), which is more hydrophobic in nature than the unmodified form of the dye and is able to permeate cell membranes much more readily. As the acetoxymethyl ester form of the dye enters the cell, the ester group is removed by cytosolic esterases, thereby trapping the dye in the cytosol. For example, carboxylic acid or phenolic groups on thallium ion indicators can be masked or protected as AM esters or acetate esters, which can allow the thallium ion indicator gain cell access (i.e., to cross over the cell membrane into the cell). “AM ester,” as used herein, refers to a compound that includes an “aectoxy” group, i.e., a CH₃C(O)OCH₂— group attached to the carboxylate oxygen to form the ester C₃C(O)OCH₂OC(O)R, where OC(O)R is a generic carboxylate. “AM ester” as used herein also refers to compounds that include an “acyloxy” group, i.e., R′C(O)OCH₂OC(O)R, where R′ is alkyl or substituted alkyl. Thus, a thallium indicator encompasses a compound that includes an AM ester or acetate ester protected derivative of a compound that is sensitive to thallium ion. In certain embodiments, thallium indicators can include a spirolactone group to aid in passage of the indicator through a live cell membrane. Once inside the cell, the spirolactone ring opens, and in the presence of sufficient thallium ion, the compound can become fluorescent.

A thallium indicator also refers to a fluorogenic compound that is non-fluorescent and becomes fluorescent in the presence of thallium ions. Thus, thallium indicators also encompass fluorogenic compounds that can demonstrate an increase in fluorescence in the presence of thallium ions.

In certain embodiments, the thallium indicator can be in the form of a salt. “Salt” refers to acceptable salts of a compound that can be derived from organic and inorganic counter ions well known in the art and include, by way of example, sodium, potassium, calcium, magnesium, ammonium, and tetraalkyl ammonium. It should also be understood that more than one thallium indicator (e.g., a combination of two or more thallium indicators) can be used in the practice of methods disclosed herein.

The optical properties of the thallium indicator can be any optical property of the luminescent dye, provided that the property can change in response to thallium ion. Examples of optical properties of the luminescent dyes include, but are not limited to intensity, frequency and polarity. Thus, in some embodiments, the intensity of the dye is detected or measured. In some embodiments, thallium ion indicator compounds are provided including a luminescent dye (e.g., fluorophore) having an optical property that can change in response to thallium ion.

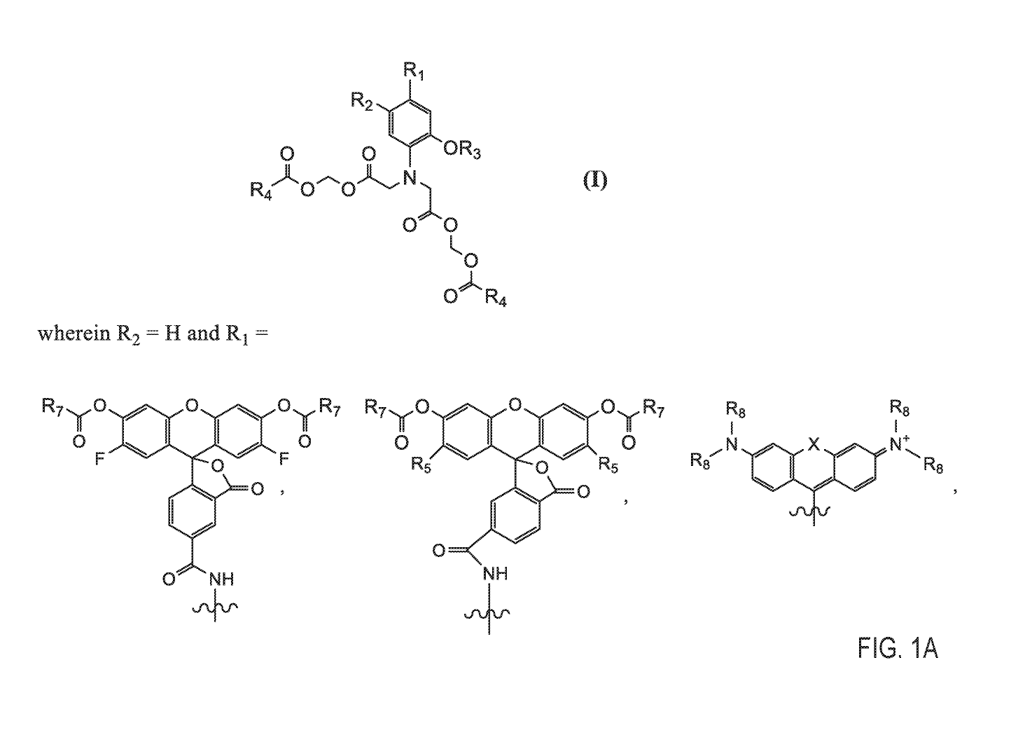

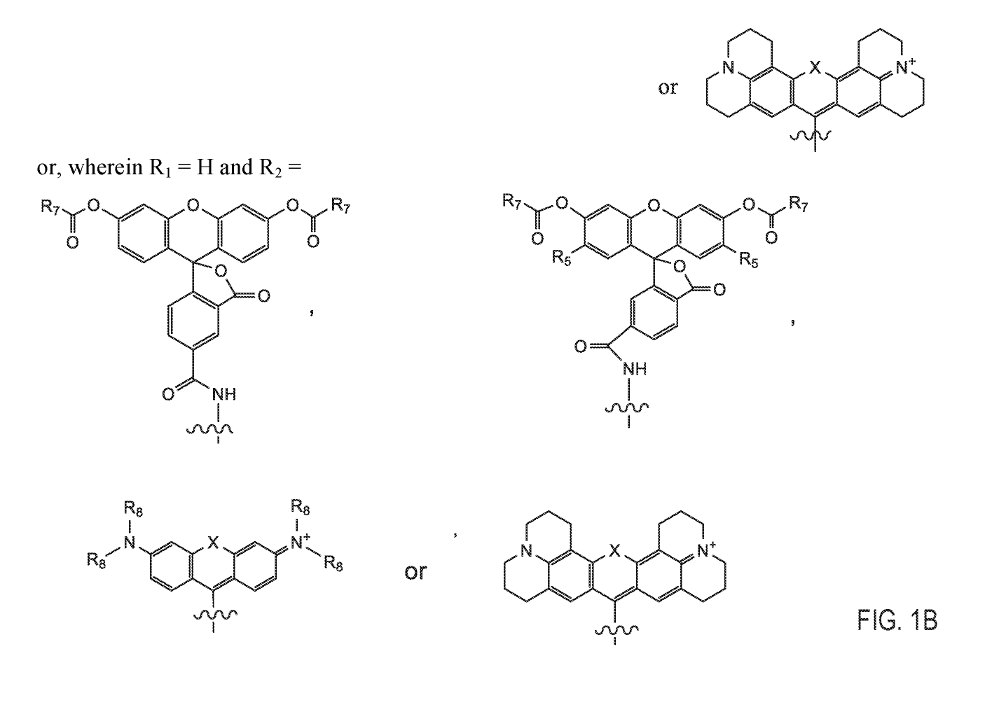

In certain embodiments, thallium indicators also can include a group a can form a complex with an ion, also referred to as an “ion-complexing moiety.” Thus, also provided herein is a complex of a thallium indicator, as disclosed herein, and a thallium ion, wherein the thallium indicator can include a group that can complex with a thallium ion. Upon binding to the thallium ion, the complex can emit light upon excitation at an appropriate spectral wavelength. Representative examples of thallium ion-complexing moieties include crown ethers, including diaryldiaza crown ethers; derivatives of 1,2-bis-(2-aminophenoxyethane)-N,N,N′,N′-tetraacetic acid (BAPTA); derivatives of 2-carboxymethoxy-aniline-N,N diacetic acid (APTRA); 2-methoxy-aniline-N,N-diacetic acid and derivative thereof, and pyridyl-based and phenanthroline metal ion chelators. A representative ion complexing group is shown in the general chemical structure (I) depicted in FIG. 1A.

In certain embodiments, thallium ion indicator compounds described herein can include a luminescent dye (e.g., fluorophore) having a property that can change in response to thallium ion and a thallium ion-complexing group. Any compound that exhibits a change in one or more of its fluorescence properties in response to binding of thallium can be used in the practice of disclosed methods. Exemplary thallium indicators include fluorescent compounds based on xanthene. Xanthene-based compounds include, for example, fluorosceins, rhodols or rhodamines. Exemplary xanthene-based compounds include fluorosceins or rhodols substituted on one or more aromatic carbons by a halogen, such as, for example, fluorine. In certain embodiments, the fluorophore is a xanthene derivative. In some embodiments, the thallium indicator is an AM ester derivative of a xanthene-based compound that contains at least one carboxylic acid or phenol. In other embodiments, the thallium indicator is a rhodol derivative or a rhodamine derivative.

The thallium ion indicator can be a fluorescent dye (e.g., an environmentally sensitive dye) or a non-fluorescent compound (e.g., a compound that associates with a thallium ion and becomes fluorescent). Thus, in one aspect, compounds are provided that include a luminescent dye (e.g., a xanthene-based dye) and a thallium ion-complexing group. In certain embodiments, the thallium ion-complexing group is a thallium ion chelator.

Thallium ion indicators including a luminescent dye and a thallium ion-complexing group can have a general structure (I) represented in FIG. 1, wherein X=O or (R₆)₂C; R₃, R₄, R₆ and R₈ are independently C₁-C₆ alkyl; R₅ is H or F; and R₇ is H, CH₃ or C₂-C₆ alkyl, and salts thereof. In compounds including R₅ substituents, one or both of R₅ can be fluorine. Fluorine-substituted fluorescent dyes can have particular advantages relative to their non-fluorinated analogues when utilized as thallium ion indicators, as disclosed herein. Specifically, thallium indicators including fluorinated dyes can possess greater photostability and have lower sensitivity to pH changes in the physiological range of 6-8, exhibit less fluorescence quenching, and possess additional advantages, such as lower pKa and higher quantum yield, while maintaining similar wavelengths of maximum absorption and emission properties relative to non-fluorinated analogues.

Fluorinated indicators having a low pKa, such as disclosed herein, can be fully ionized at neutral pH and, therefore, can experience maximal fluorescence upon binding thallium ion. Because the basal fluorescence of fluorinated dyes is typically lower than that exhibited by the non-fluorinated analogue, this combination of low basal fluorescence and maximal thallium ion binding for fluorinated thallium indicators can result in the production of very large signal to noise windows when such compounds are implemented in the thallium ion detection assays described herein. Non-fluorinated derivatives that are only partially ionized at neutral pH, however, may exhibit a substantially smaller signal to noise window when utilized in the assays described herein. As mentioned above, fluorinated dyes also are relatively insensitive to pH changes. When utilized in the context of thallium ion detection, a dye's pH sensitivity bias or alter the report from the dye. Advantageously, the signal specificity for a thallium analyte is minimally affected by interference due to pH changes using thallium indicators including fluorinated dyes, such as described herein. An additional benefit of implementing fluorinated compounds for thallium ion detection is that such indicators are effective over a broad range of dye loading concentrations. For example, the fluorogenic response for fluorinated compounds in the presence of thallium ions can remain relatively constant over range of loading buffer concentrations (e.g., about 0.3 to 30 μM) when utilized in the thallium ion detection assays disclosed herein. In comparison, overloading or underloading of non-fluorinated indicators can compromise the activity of the indicator under the same assay conditions, thus restricting their use to a narrower range of concentrations.

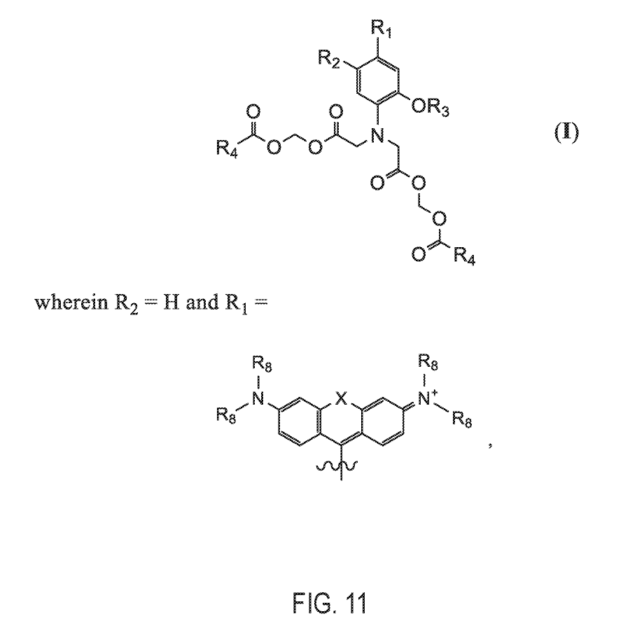

In certain embodiments, thallium sensitive compound has a structure represented in FIG. 11, wherein X=O or (R₆)₂C; R₃, R₄, R₆ and R₆ are independently C₁-C₆ alkyl; R₅ is H or F; and R₇ is H, CH₃ or C₂-C₆ alkyl, with the exception that all four R₈ groups cannot be C1 alkyl (i.e., methyl).

Also provided herein are compositions that include compounds having structures as represented in FIG. 1A and FIG. 1B, wherein the compounds are dissolved in an aqueous medium, such as a buffer or water.

Although the compounds described herein are described for use in the detection of thallium ions, the described compounds also can be sensitive to other types of metal ions. Particularly relevant are those metal ions that are present in biological systems or systems relevant to the study of metabolism or toxicology, such as, e.g., Mg²⁺, Fe²⁺Zn²⁺, Pb²⁺, Cd²⁺ and the like. Thallium sensitive agents disclosed herein are typically insensitive to the presence of calcium ions.

The described compounds can be used in assays involving detection of thallium or other metal ions in applications apart from hos specifically disclosed herein. For example, the present compounds can be utilized to bind, detect, quantitate, monitor and further analyze metal ions including, but not limited to thallium ion. An exemplary method for binding a target metal ion (e.g., Zn²⁺) in a sample includes contacting the sample with a compound, as disclosed herein, to form a contacted sample; and, incubating the contacted sample for a sufficient amount of time to allow the compound to chelate the target metal ion whereby the metal ion is bound. The method further includes detecting the target metal ion, wherein the sample illuminated with an appropriate wavelength whereby the target metal ion is detected.

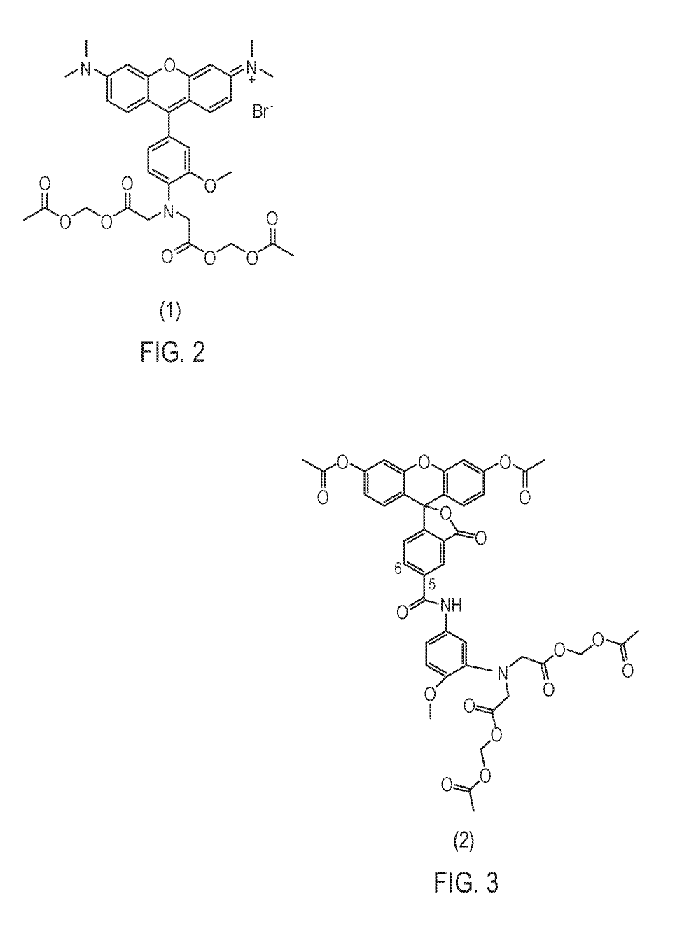

Representative thallium ion sensitive compounds provided herein include those depicted, e.g., in FIG. 2-FIG. 9. Certain compounds provided herein can exist as different structural isomers. For example, thallium sensitive compounds can include a parent structure having two e substituents, where one or more substituents occupy a different position to form compounds with different chemical structures. By way of illustration, a compound having a general structure (1) (see, FIG. 1A) can include an aromatic ring bearing two or more substituents (R₁ and R₂) on the benzene ring, wherein substituents R₁, R₂, R₃ and R₄ are as disclosed herein.

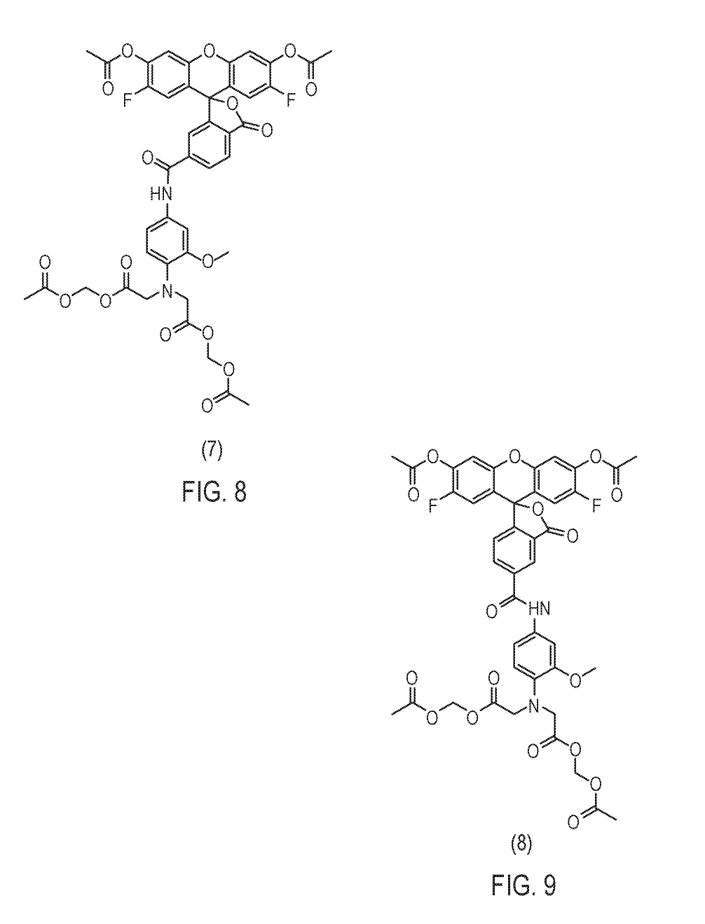

Referring to FIG. 5 and FIG. 8, two compounds are shown having the same parent structure (I) and the same xanthene fluorophore unit. As disclosed herein, either R₁ or R₂ can be H, if R₁ is H, R₂ contains a fluorophore moiety and if R₂ is H, R₁ contains a fluorophore moiety. Various structural isomers are described herein. By way of illustration, in Compound (4), R₁ is H; whereas in Compound (7), R₂ is H. As a result, the amide nitrogen in Compound (4) is positioned in a meta orientation relative to the nitrogen atom of the bis(acetoxymethyl) 2,2′-azanediyldiacetate substituent, and in Compound (7), the amide nitrogen is positioned in a para orientation relating to the nitrogen atom of the bis(acetoxymethyl) 2,2′-azanediyldiacetate substituent. Because compounds (4) and (7) differ only in he relative positioning of substituents on a benzene ring, these two compounds are structural isomers of each other.

Surprisingly, it was found that the performance of certain thallium-sensitive compounds having the identical parent structure varied considerably depending on which structural isomer was utilized according to the thallium detection methods disclosed herein. For certain sets of structural isomers, the difference in performance under the same assay conditions was dramatic, e.g., two-fold or greater, between meta and para isomers of the identical parent structure (see, Table 1). In general, the fold increase of fluorescence signal over baseline for the para isomers was significantly higher than when measured for the meta isomer.

The thallium ion sensitive fluorescent agents can be loaded into the cell by contacting the cells with a loading buffer comprising the dye or a membrane-permeable derivative of the dye. A loading buffer a solution that loads thallium ion indicator (e.g., a thallium ion sensitive fluorescent agent) into a cell. Loading the cells with the dye may be further facilitated by a snore hydrophobic form of the dye. For example, as the acetoxymethyl ester form of the dye enters the cell, the ester group is removed by cytosolic esterases, thereby trapping the dye in the cytosol.

In one specific embodiment where a fluorescent thallium ion sensitive agent is used, the excess fluorescent compound can be removed by using a sufficient amount of an extracellular quencher. The use of extracellular quencher removes the need to wash unloaded thallium ion sensitive fluorescent agent from the cells. The extracellular quenchers are preferably not cell permeant and can be light absorbing fluorescent compounds having a fluorescence that can be easily separated from that of the thallium ion sensitive fluorescent agent. The absorption spectrum of the extracellular quenchers significantly absorbs the emission of the thallium ion sensitive fluorescent agent. The extracellular quenchers typically have a chemical composition that prevents their passage into the cells, and, generally speaking, the quenchers should be charged or be very large compounds. The concentration range for extracellular quenchers may range from micromolar to millimolar concentrations, depending on their light absorbing properties. Types of extracellular quenchers that can be used include, but are not limited to, tartrazine and amaranth, or a mixture of such quenchers, or other quenchers known to those skilled in the art.

The loading buffer can also include chloride ions. In one embodiment, the loading buffer comprises a detectable amount of chloride ions. In another embodiment, the loading buffer is chloride-free. One of the solutions provided is to allow the use of chloride in the loading buffer and in the cell media, prior to stimulating the cells with thallium ion. In general, the source of chloride in buffers is usually from the NaCl salt, but the chloride can be from any source if present. The chloride, if present in the buffers, e.g., the loading buffer or the washing buffer, can be at virtually any centration, because the disclosed methods are not dependent upon the absence of chloride. In one specific embodiment, the loading buffer comprises chloride that is present in physiological relevant concentrations, i.e., ˜10 mM. Other concentrations of chloride may also be used, where one of skill in the art can readily determine the levels of chloride that are acceptable.

After the loading buffer is provided to the cells, the stimulus buffer is added to the cells to stimulate thallium ion into or out of the cells. The stimulus buffer typically includes thallium ion. A stimulus buffer is a solution that activates the ion channel, channel-linked receptor or ion transporter (e.g., agonist). Some ion channels/transporters may be constitutively active and thus would not require a “stimulus” in addition to the thallium ion tracer. For channels that require a stimulus, that stimulus may be ligand (a molecule that binds to the channel or channel linked receptor and activates the same (an agonist). A stimulus might also be a change in membrane potential for voltage-gated channels. Typically voltage-gated. channels are activated by either direct electrical stimulation with electrodes or by using a stimulus solution that contains an ionic composition that will cause depolarization (such as high external potassium). In addition, thallium ions can also act as a stimulus for voltage-gated channels. In such a case, thallium ions can act as both a “tracer” and a depolarizing stimulus. In an influx assay, thallium ions can be added just before, during, or after the addition of a stimulus.

The methods disclosed herein can include stimulus buffers that are selected based on the type of ion channel, channel-linked receptor or ion transporter used in the method. Selecting an appropriate stimulus solution and ion channel, channel-linked receptor or ion transporter-activating reagent, is within the capability of one skilled in the art. In one embodiment, the stimulus buffers include a buffer that does not include reagents that activate the ion channel, such that the ion channels, the channel-linked receptors or the ion transporters remain substantially at rest. In this embodiment, the stimulus solution includes reagents that do not activate the ion channel, channel-linked receptor or ion transporter of interest but facilitate activation of ion channel, channel-linked receptor or ion transporter when a modulating reagent is added to the cells to initiate the assay.

The stimulus solution selected for use with voltage-dependent ion channels, e.g., the N-type calcium channel or KCNQ2 channel, depends upon the sensitivity of the ion channel to the resting potential of the cell membrane. For methods using these voltage-dependent ion channels, the stimulating solution may include activating reagents that serve to depolarize the membrane, e.g., ionophores, valinomycin, and the like.

A stimulus buffer selected for use with some voltage-dependent ion channels for activation by depolarization of the cell membrane includes potassium salt at a concentration such that the final concentration of potassium ions in the cell-containing well is in the range of about 10-150 mM, e.g., 50 mM KCl. In addition, voltage-dependent ion channels may also be stimulated by an electrical stimulus.

The stimulus buffer selected for use with channel-linked receptors and ligand-gated ion channels depends upon ligands that are known to activate such receptors. For example, nicotinic acetylcholine receptors are known to be activated by nicotine or acetylcholine; similarly, muscarinic acetyl choline receptors may be activated by addition of muscarine or carbamylcholine. The stimulating buffer for use with these systems may include nicotine, acetylcholine, muscarine or carbamylcholine.

Thallium ion in the stimulus buffer can be in any form, but it will primarily be in the form of a salt, thus providing thallium ions. The thallium ion salts for use in thallium ion solutions used in the methods described herein include those that are water soluble, such as but not limited to, Tl₂SO₄, Tl₂CO₃, TlCl, TlOH, TlOAc, TlNO₃ salts and the like.

The transport of thallium ion sensitive agents and thallium ions into cells is followed by an increase or decrease in the signal of the thallium ion sensitive agent. Thallium ions can move through open channels along their concentration gradient and change the intensity of dye fluorescence inside the cell, resulting in the recorded signals. Activation of the ion channel enhances the rate of influx of thallium ions (resulting in a change in the fluorescence of the thallium ion sensitive fluorescent compound and inhibition decreases the rate of influx of thallium ions (resulting in no or little change in the fluorescence of the thallium ion sensitive fluorescent agent). Generally the fluorescence remains the sane if no thallium ion is bound to it.

At least one optical property of the thallium indicator is detected or measured in the methods described herein. As mentioned earlier, when fluorescent dyes are utilized, any optical property of the fluorescent dye can be measured or detected to determine thallium ion influx or efflux. Examples of optical properties of fluorescent dyes include, but are not limited to, intensity, polarity and frequency the luminescence. If non-fluorescent dyes are used as the detecting agent, the optical properties of the agent to be detected can be also be intensity, polarity and frequency. In one specific aspect, measuring or detecting the optical properties of the agent includes measuring the optical density of the cells themselves, when, for example, the agent reacts with thallium ion to form a product or precipitant within the cell itself that may increase the optical density of the cell itself.

The fluorescence of the thallium ion sensitive agent can be measured by devices that detect fluorescent signals, such as but not limited to spectrophotometers, microscopes and the like. In one specific embodiment, the fluorescence of the dyes is detected and/or measured using a standard 96-well plate reader. Another type of device is a Fluorometric Image Plate Reader (FLIPR) device (Molecular Devices Corp., Sunnyvale, Calif.), where fluorescence is recorded at a rate of up to 1 Hz, before, during, and after addition of thallium ions, and addition of candidate ion channel, channel-linked receptor or ion transporter modulators. Examples of devices used for non-adherent cells include FLIPR. Additional examples of devices and methods used to detect or measure the optical properties of thallium sensitive agents include, but are not limited to, light microscopy, confocal microscopy, fluorescence microscopy and flow cytometry.

In one embodiment, the activity of channel-linked receptors is determined, where the activation of the receptor initiates subsequent intracellular events that lead to the modulation of ion channel activity.

Also provided are methods for identifying compounds that modulate ion channel, channel-linked receptor, or ion transporter activity. Essentially any chemical compound can be used as a potential modulator in the assays provided herein. In specific embodiments, the candidate compounds can be dissolved in aqueous or organic (especially DMSO-based) solutions. It will be appreciated by those of skill in the art that there are many commercial suppliers of chemical compounds, including Sigma Chemical Co. (St. Louis, Mo.) and Fluka Chemika-Biochemica Analytika (Buchs, Switzerland). Thus, if the ion channel is blocked by the candidate channel modulator and thallium ion influx is inhibited, little or no change in fluorescence will be detected.

Also provided are methods for measuring the efflux of ions. The methods of measuring thallium ion influx are described herein. The efflux assays can use the same cells as in the influx assays, and are loaded with a signal generating thallium ion sensitive fluorescent agent, as described herein, such as BTC. The cells are contacted with thallium ion to load the cells. One embodiment provides contacting the cells with thallium ions for approximately 15 minutes. The cells are washed to remove excess thallium ions and assayed using the same instrument to detect changes in signal as used in the influx assay, e.g., FLIPR. The assay channels are stimulated to open by the addition of any one of a number of ligands, or by changing the membrane potential of the cell, such as by changing the potassium concentrations, to permit efflux of ions through the ion channels. For example, an efflux would result in a decrease in fluorescence of the indicator. The other compounds, such as control compounds, can be the same as used in the influx assays. The same conditions are applied as for the influx assay described herein, except the cells are preloaded with thallium ions as described above, and washed to remove excess thallium ions.

The disclosed methods also can be adapted to high-throughput screening (HTS) methods, such that candidate ion channel modulators can be screened on a large scale. High-throughput screening assays are known, and can employ microtiter plates or pico-nano- or micro-liter arrays.

The high-throughput methods can be performed using whole cells expressing ion channels, ion channel and channel-linked receptors or ion transporters of interest, by practicing the instant methods on microtiter plates or the like. The cells can be cultured under adherent or non-adherent conditions. The candidate modulators are added to the cells and then the stimulus buffer(s) are added to the cells and, for example, fluorescence is detected. The change in the detectable signal would indicates the effect of the channel modulators in a particular well on a plate.

The assays disclosed herein are designed to permit high throughput screening of large chemical libraries, e.g., by automating the assay steps and providing candidate modulatory compounds from any convenient source to assay. Assays which are run in parallel on a solid support, e.g., microtiter formats on microtiter plates in robotic assays, are well known. Automated systems and methods for detecting and/or measuring changes in optical detection are well known in the art.

High throughput screening methods can include providing a combinatorial library containing a large number of potential therapeutic modulating compounds. Preparation and screening of combinatorial chemical libraries is well known to those of skill in the art. A combinatorial chemical library is a collection of diverse chemical compounds generated by using either chemical synthesis or biological synthesis, to combine a number of chemical building blocks, such as reagents. For example, a linear combinatorial chemical library, such as a polypeptide library, is formed by combining a set of amino acids in virtually every possible way for a given compound length, i.e., the number of amino acids in a polypeptide compound. Millions of chemical compounds can be synthesized through such combinatorial mixing of chemical building blocks.

The compounds provided herein are highly sensitive indicators that quantitatively detect thallium ions even at very low concentrations under physiologic-relevant conditions. In addition, compounds are provided the emit light that does not interfere with other fluorescent components in HTS assays, such as, e.g., green fluorescent proteins (GFP). Because these compounds can absorb and emit light outside of the FITC/green optical channel, these compounds can he used in HTS assays to emit light over a range of visible wavelengths in response to the presence of metal ions such a thallium and can be used effectively in monitoring the activity of ion channels.

Also provided herein are kits for detecting the activity of ion channels in a cell. In one particular embodiment, the kits comprise a thallium ion indicator, as disclosed herein, the assay buffer. The assay buffer can include chloride, and the stimulus buffer. The individual components of the buffers and the dyes can be lyophilized or stored in some other dehydrated form, where the individual can hydrate the components into stock or working solutions. The kits can contain virtually any combination of the components set out above or described elsewhere herein. As one skilled in the art would recognize, the components supplied with kits can vary with the intended use for the kits. The kits also include instructions for use in the methods disclosed herein. Thus, kits can be designed to perform various functions set out in this application and the components of such kits will vary accordingly.

The following examples are included to demonstrate certain embodiments of the invention. It should be appreciated by those of skill in the art that the techniques disclosed in the examples which follow represent techniques discovered by the inventor(s) to function well in the practice of the invention, and thus can be considered to constitute preferred modes for its practice. However, those of skill the art should, in light of the present disclosure, appreciate that many changes can be made in the specific embodiments which are disclosed and still obtain a like or similar result without departing from the scope of the invention.

### EXAMPLES

### Example 1

### Synthesis of bis(acetoxymethyl) 2,2′-((4-(3′,6′-diacetoxy-2′,7′-difluoro-3-oxo-3H-spiro[isobenzofuran-1,9′-xanthene]-6-carboxamide)-2- methoxyphenyl)azanediyl)diacetate

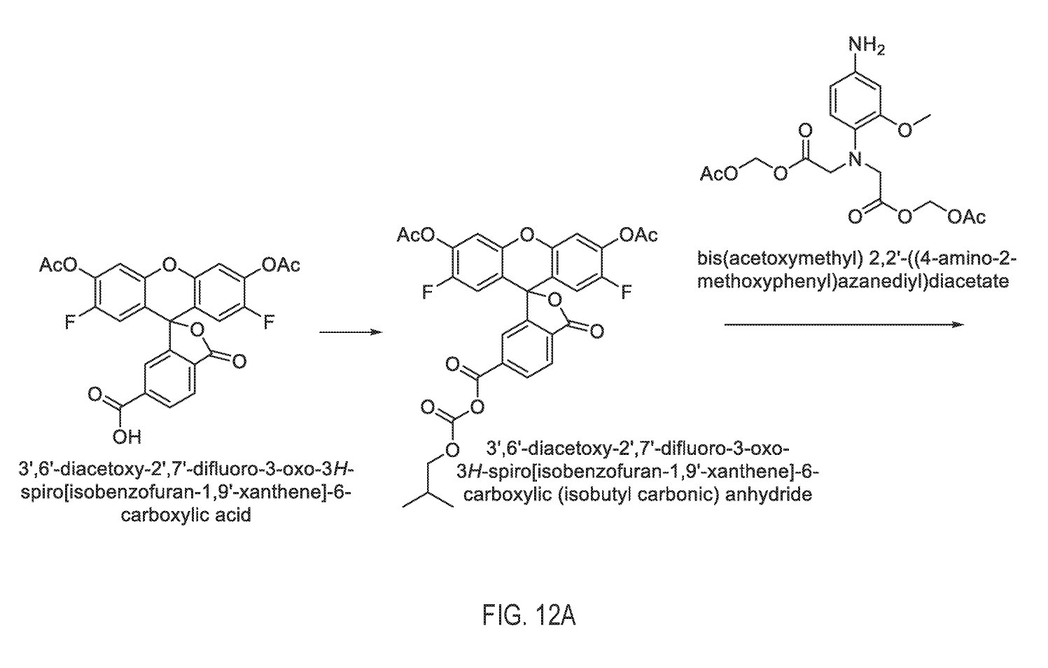

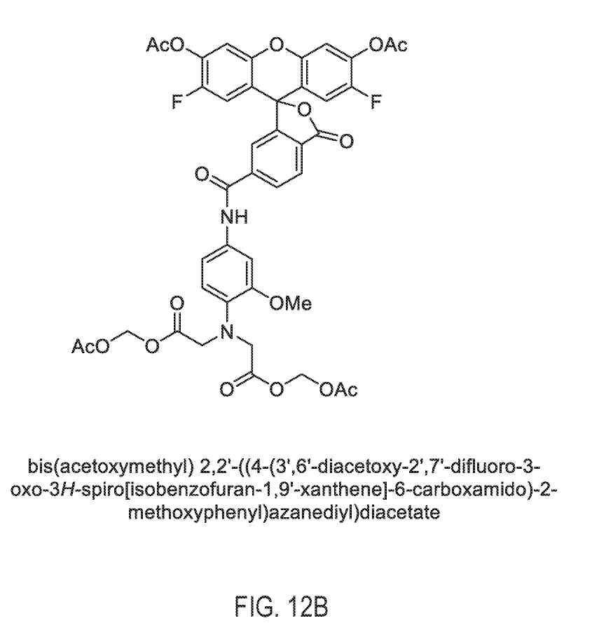

The reaction scheme for the synthesis of bis(acetoxymethyl) 2,2′-((4-(3′,6′-diacetoxy-2′,7′-difluoro-3-oxo-3H-spiro[isobenzofuran-1,9′-xanthene]-6- carboxamido)-2-methoxyphenyl)azanediyl)diacetate is shown in FIG. 12A and FIG. 12B. 3′,6′-diacetoxy-2′,7′-difluoro-3-oxo-3H-spiro[isobenzofuran-1,9′-xanthene]-6-carboxylic acid (236 mg, 0.475 mmol) was suspended in 15 mL of dry DCM and the suspension was cooled in ice water bath. Triethylamine (66 μL, 0.47 mmol) was added to the suspension followed by isobutyl chloroformate (73 μL, 0.56 mmol) and the resulting solution was stirred for 20 min in cooling bath. After that the solution was concentrated and in vacuum to provide 3′,6′-diacetoxy-2′,7′-difluoro-3-oxo-3H-spiro[isobenzofuran 1,9′-xanthene]-6-carboxylic (isobutyl carbonic) anhydride (280 mg, 100%). Bis(acetoxymethyl) 2,2′-((4-amino-2-methoxyphenyl)azanediyl)diacetate (172 mg, 0.432 mmol) was dissolved in 10 mL of dry DMF and solution added to the flask containing 3′,6′-diacetoxy-2′,7′-difluoro-3-oxo-3H-spiro[isobenzofuran 1,9′-xanthene]-6-carboxylic (isobutyl carbonic) anhydride (280 mg, 0.47 mmol) and the resulting solution was stirred for 20 min at RT. The reaction mixture was concentrated in vacuum. The residue was dissolved in 100 mL of EtOAc and washed with 5% HCl (30 ml), water (2×30 mL), brine (30 mL), dried over Na2SO4 and evaporated. The crude material was purified on silica gel column using EtOAc—hexane gradient (0-60%). After the combined fractions were evaporated, the material was re-dissolved in 1 mL of EtOAc and precipitated with 50 mL of hexane. The precipitate was filtered and dried in vacuum to provide bis(acetoxymethyl) 2,2′-((4-(3′,6′-diacetoxy-2′,7′-difluroro-3-oxo-3H-spiro[isobenzofuran-1,9′-xanthene]-6-carboxamido)-2-methoxyphenyl)azanediyl)diacetate (224 mg, 59%) (Compound 7) (see, FIG. 8)

The same method was used to prepare non-fluorinated analogue, Compound. 6, with the exception that bis(acetoxymethyl) 2,2′-((4-(3′,6′-diacetoxy-3-oxo-3H-spiro[isobenzofuran-1,9′-xathene]-6-carboxamido)-2-methoxyphenyl)azanediyl)diacetate was used in the synthesis (see. FIG. 7).

### Example 2

### Synthesis of bis(acetoxymethyl) 2,2′-(4-(3′,6′-diacetoxy-2′,7′-difluoro-3-oxo-3H-spiro[isobenzofuran-1,9′-xanthene]-5-carboxamido)-2- methoxyphenyl)azanediyl)diacetate

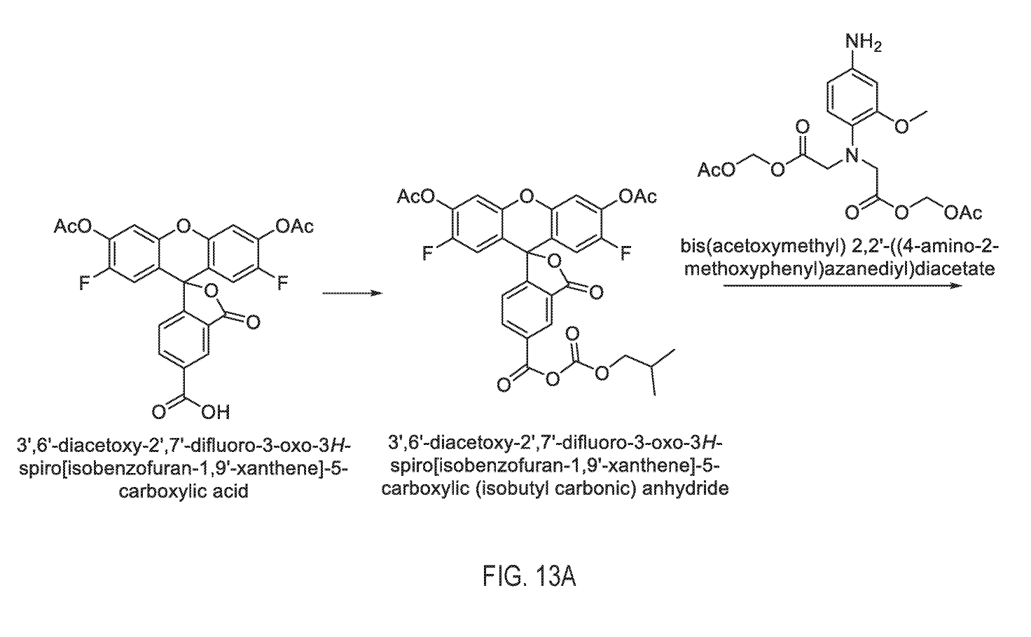

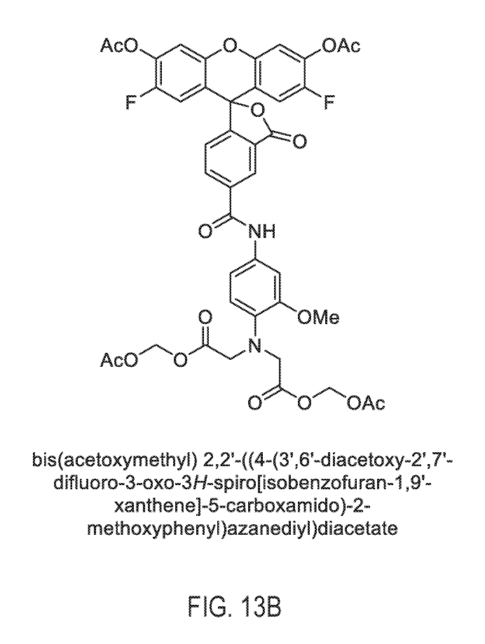

The reaction scheme for the synthesis of bis(acetoxymethyl) 2,2′-((4-(3′,6′-diacetoxy-2′,7′-difluoro-3-oxo-3H-spiro[isobenzofuran-1,9′-xanthene]-5- carboxamido)-2-methoxyphenyl)azanediyl)diacetate is shown in FIG. 13A and FIG. 13B, 3′,6′-diacetoxy-2′,7′-difluoro-3-oxo-3H-spiro[isobenzofuran-1,9′-xanthene]-5-carboxylic acid (283 mg, 0.570 mmol) was suspended in 20 mL of dry DCM and the suspension was cooled in ice water bath. Triethylamine (79 μL, 0.56 mmol) was added to the suspension followed by isobutyl chloreformate (88 μL, 0.67 mmol) and the resulting solution was stirred for 20 min in cooling bath. After that the solution was concentrated and in vacuum to provide 3′,6′-diacetoxy-2′,7′-difluoro-3-oxo-3H-spiro[isobenzofuran-1,9′-xanthene]-5-carboxylic (isobutyl carbonic) anhydride (336 mg, 100%). Bis(acetoxymethyl) 2,2′-((4-amino-2-methoxyphenyl)azanediyl)diacetate (206 mg, 0.518mmol) was dissolved in 12 mL of dry DMF and solution added to the flask containing 3′,6′-diacetoxy-2′,7′-difluoro-3-oxo-3H-spiro[isobenzofuran 1,9′-xanthene]-5-carboxylic (isobutyl carbonic) anhydride (336 mg, 0.564 mmol) and the resulting solution was stirred for 20 min at RT. The reaction mixture was concentrated in vacuum the residue dissolved in 120 mL of EtOAc, washed with 5% HCl (40 ml), water (2×40 mL), brine (40 mL), dried over Na₂SO₄ and evaporated. The rude material was purified on silica gel column using EtOAc—hexane gradient (0-60%). After the combined fractions were evaporated, the material was re-dissolved in 1.5 mL of EtOAc and precipitated with 70 mL of hexane. The precipitate was filtered and dried in vacuum to provide bis(acetoxymethyl) 2,2′-((4-(3′,6′-diacetoxy-2′,7′-difluoro-3-oxo-3H-spiro[isobenzofuran-1,9′-xanthene]-5-carboxamido)-2-methoxyphenyl)azanediyl)diacetate (278 mg, 61%) (Compound 8) (see, FIG. 9).

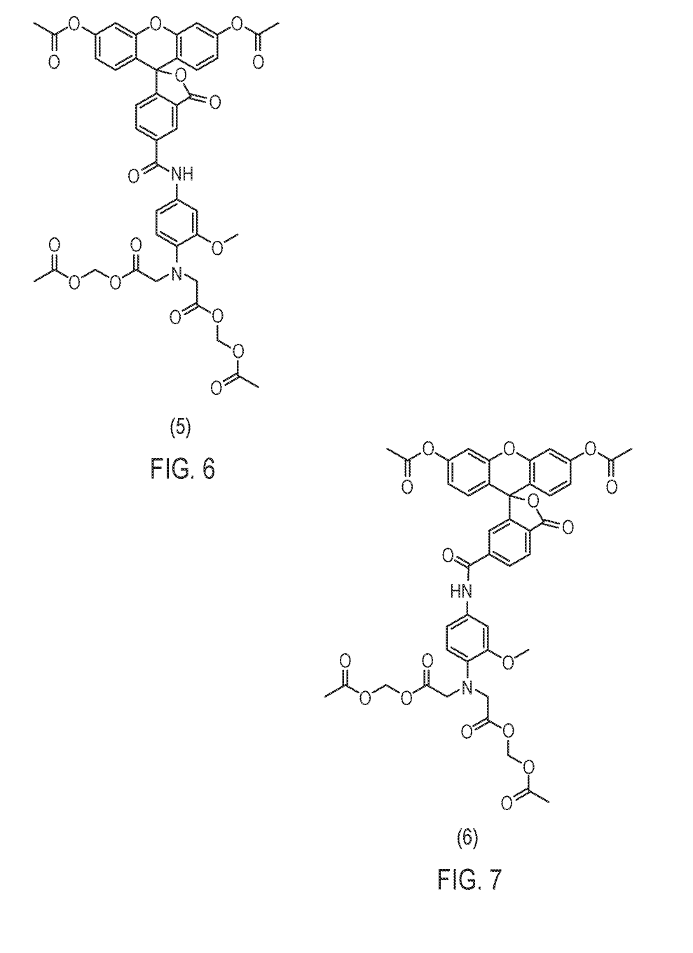

The same method was used. to prepare non-fluorinated analogue, Compound. 5, with the exception that bis(acetoxymethyl) 2,2′-((4-(3′,6′-diacetoxy-3-oxo-3H-spiro[isobenzafuran-1,9′-xanthene]-5-carboxamido)-2-methoxyphenyl)azanediyl)diacetate was used in the synthesis (see, FIG. 6).

### Example 3

Synthesis of bis(acetoxymethyl)-2,2′-((5-amino-2-methoxyphenyl)azanediyl)diacetate

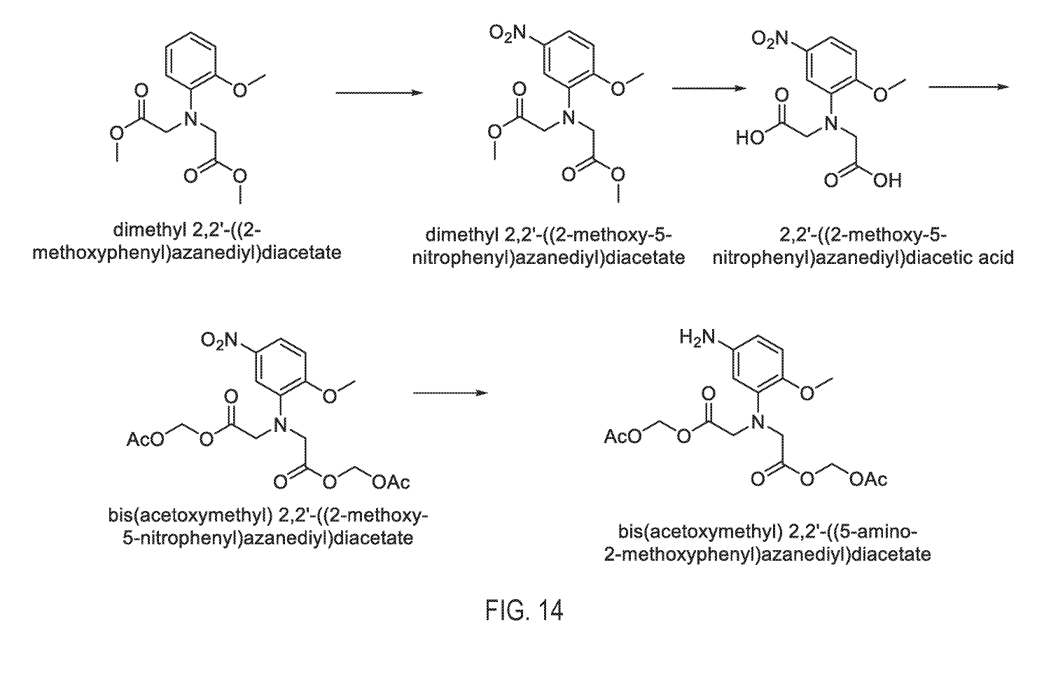

The reaction scheme for the synthesis of bis(acetoxymethyl)-2,2′-((5-amino-2-methoxyphenyl)azanediyl)diacetate is shown in FIG. 14. Dimethyl 2,2′-((2-methoxyphenyl)azanediyl)diacetate (315 mg; 1.178 mmol) was dissolved in 96% sulfuric acid (0.86 mL; 15 mmol) and solution cooled in ice water bath for 15 min. Powdered potassium nitrate (119 mg; 1.17702 mmol) was added to the mixture portionwise during 15 min. The reaction mixture was stirred for 2 hrs in cooling bath, then quenched with ice and the product extracted with EtOAc (3×50 mL). The combined extract was washed with sat. NaHCO3, water, brine, dried over Na₂SO₄ and evaporated. The crude material was purified on silica gel column with ethyl acetate—hexane gradient (0-40%) to obtain dimethyl 2,2′-((2-methoxy-5-nitrophenyl)azanediyl)diacetate (220 mg, 60%). Dimethyl 2,2′-((2-methoxy-5-nitrophenyl)azanediyl)diacetate (220 mg; 0.7045 nmol) was suspended in the mixture of 1 mL of dioxane and 1 of MeOH. Potassium. hydroxide (1M, 4 mL) was added to this suspension and reaction mixture was stirred for 2.5 hrs at RT. The resulting solution was concentrated on rotary evaporator and the residue (˜3 mL) was added dropwise to 10 mL of 3 M HCl solution with stirring. Yellow precipitated was stirred for 30 min, filtered and the solid dried at 40° C. for 20 hrs to obtain 2,2′-((2-methoxy-5-nitrophenyl)azanediyl)diacetic acid (182 mg, 91%). 2,2′-((2-methoxy-5-nitrophenyl)azanediyl)diacetic acid (180 mg; 0.63331 mmol) was dissolved in ˜2 mL of dry DME N,N,N-dilsopropylethylamine (0.33 mL, 1.9 mmol) was added to the solution, followed by adding bromomethyl acetate (0.22 mL, 2.2 mmol). The reaction mixture was stirred for ˜20 h at RT and diluted with EtOAc (100 mL). The resulting suspension was washed with 5% HCl, (20 mL), water (20 mL), brine (20 mL), dried over Na₂SO₄ and evaporated. The crude material was purified on silica gel column with EtOAc—hexane gradient (0-65&) to obtain bis(acetoxymethyl) 2,2′-((2-methoxy-5-nitrophenyl)azanediyl)diacetate (271 mg, 100%). Bis(acetoxymethyl) 2,2′-((2-methoxy-5-nitrophenyl)azanediyl)diacetate (40 g, 93 μmol) was dissolved in 5.0 mL of dry DMF in 250 mL hydrogenation flask; 10 mg of 10% Pd on carbon was added to the solution and the flask was shaken for 45 min at 40 psi of hydrogen. The catalyst was filtered off and solution evaporated to dryness to provide bis(acetoxymethyl) 2,2′-((5-amino-2-methoxyphenyl)azanediyl)diacetate (37 mg, 100%).

### Example 4

Synthesis of bis(acetoxymethyl) 2,2′-((5-(3′,6′-diacetoxy-2′,7′-3-oxo-3H-spiro[isobenzofuran-1,9′-xanthene]-6-carboxamido)-2- methoxyphenyl)azanediyl)diacetate

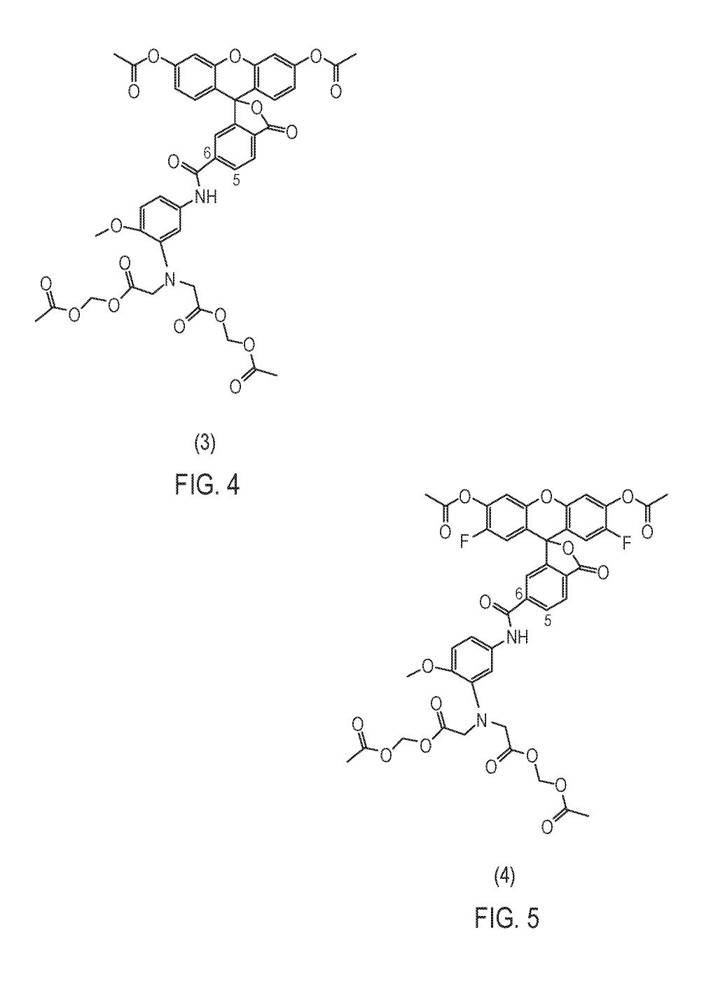

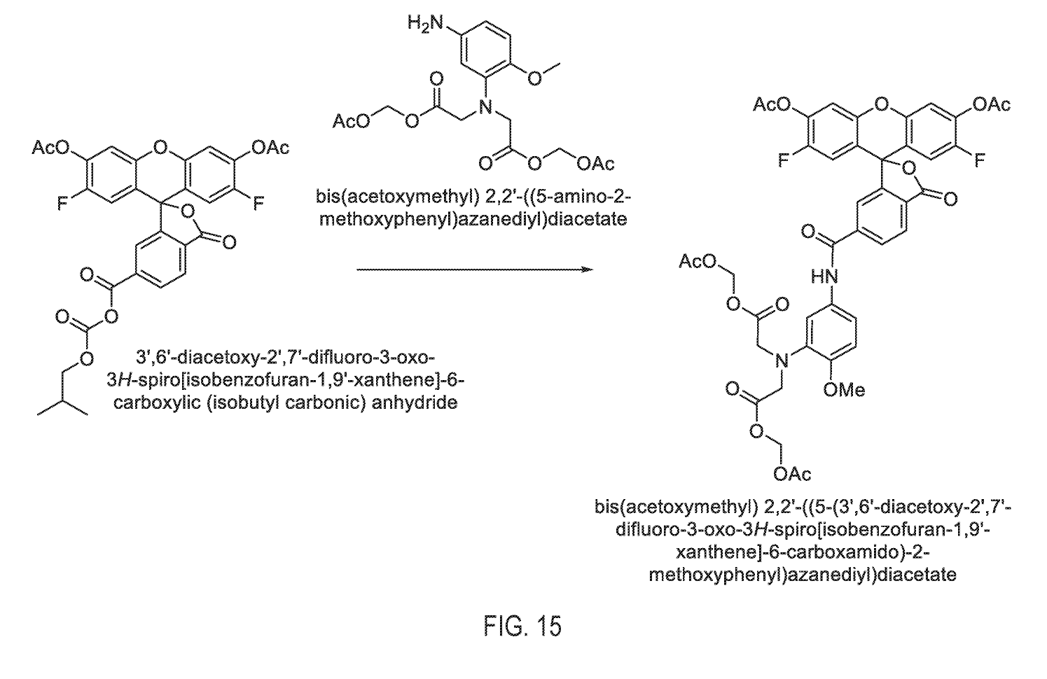

The reaction scheme for the synthesis of his(acetoxymethyl) 2,2′-((5-(3′,6′-diacetoxy-2′,7′-difluoro-3-oxo-3H-spiro[isobenzofuran-1,9″-xathene]-6- carboxamido)-2-methoxyphenyl)azanediyl)diacetate is shown in FIG. 15. Bis(acetoxymethyl) 2,2′-((5-amino-2-methoxyphenyl)azanediyl)diacetate (103 mg, 0.259 mmol) was dissolved in 8 mL of dry DMF and solution added to the flask containing 3′,6′-diacetoxy-2′,7′-difluoro-3-oxo-3H-spiro[isobenzofuran1,9′-xanthene]-6-carboxylic (isobutyl carbonic) anhydride (168 mg, 0.282 mmol) and the resulting solution was stirred for 2.0 min at RT. The reaction mixture was concentrated in vacuum the residue dissolved in 80 mL of EtOAc, washed with 5% HCl (30 ml), water (2×30 mL), brine (30 mL), dried over Na₂SO₄ and evaporated. The crude material was purified on silica gel column using EtOAc—hexane gradient (0-60%). After the combined fractions were evaporated, the material was re-dissolved in 1 mL of EtOAc and precipitated with 50 mL of hexane. The precipitate was filtered and dried in vacuum to provide bis(acetoxymethyl) 2,2′-((5-(3′,6′-diacetoxy-2′,7′-difluoro-3-oxo-3H-spiro[isobenzofuran-1,9′-xanthene]-6-carboxamido)-2- methoxyphenyl)azanediyl)diacetate (113 mg, 50%) (Compound 4) (see, FIG. 5). The same method was used to prepare non-fluorinated analogue, Compound 3, with the exception that bis(acetoxymethyl) 2,2′-((5-(3′,6′-diacetoxy-3-oxo-3H-spiro[isobenzofuran-1,9′-xanthene]-6-carboxamido)-2-methoxyphenyl)azanediyl)diacetate was used in the synthesis (see, FIG. 4).

### Example 5

### Synthesis of bis(acetoxymethyl) 2,2′-((5-(3′,6′-diacetoxy-2′,7′-difluoro-3-oxo-3H-spiro[isobenzofuran-1,9′-xanthene]-5-carboxamido)-2- methoxyphenyl)azanediyl)diacetate

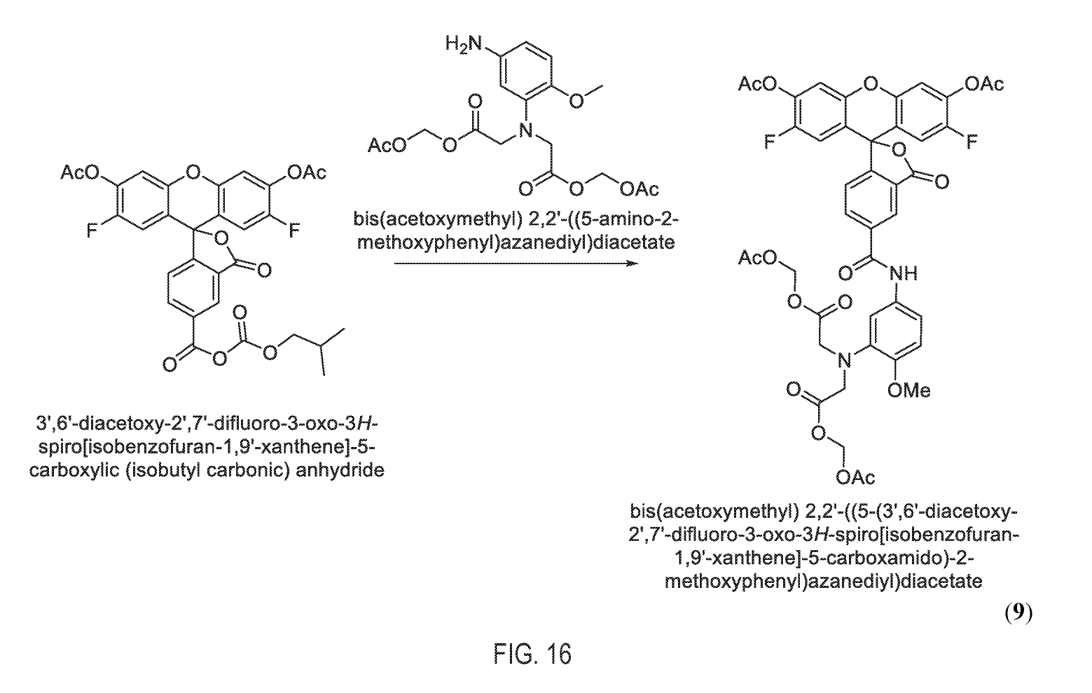

The reaction scheme for the synthesis of bis(acetoxymethyl) 2,2′-((5-(3′,6′-diacetoxy-2′,7′-difluoro-3-oxo-3H-spiro[isobenzofuran-1,9′-xanthene]-5- carboxamido)-2-methoxyphenyl)azanediyl)diacetate is shown in FIG. 16. Bis(acetoxymethyl) 2,2′-((5-amino-2-methoxyphenyl)azanediyl)diacetate (134 mg, 0.337 mm ol) was dissolved in 10 mL of dry DMF and solution added to the flask containing 3′,6′-diacetoxy-2′,7′-difluoro-3-oxo-3H-spiro[isobenzofuran1,9′-xanthene]-5-carboxylic (isobutyl carbonic) anhydride 18 mg, 0.367 mmol) and the resulting solution was stirred for 20 min at RT. The reaction mixture was concentrated in vacuum the residue dissolved in 100 mL of EtOAc, washed with 5% HCl (40 ml), water (2×40 mL), brine (40 mL), dried over Na₂SO₄ and evaporated. The rude material was purified on silica gel column using EtOAc—hexane gradient (0-60%). After the combined fractions were evaporated, the material was re-dissolved in 1 mL of EtOAc and precipitated with 50 mL of hexane. The precipitate was filtered and dried in vacuum to provide bis(acetoxymethyl) 2,2′-((5-(3′,6′-diacetoxy-2′,7′-difluoro-3-oxo-3H-spiro[isobenzofuran-1,9′-xanthene]-5-carboxamido)-2- methoxyphenyl)azanediyl)diacetate (162 mg, 55%) (Compound 9). The same method was used to prepare non-fluorinated analogue, Compound 2, with the exception that bis(acetoxymethyl) 2,2′-((5-(3′,6′-diacetoxy-3-oxo-3H-spiro[isobenzofuran-1,9′-xanthene]-5- carboxamido)-2-methoxyphenyl)azanediyl)diacetate was used in the synthesis.

### Example 6

### Synthesis of dimethyl-2,2′-((4-formyl-2-methoxyphenyl)azanediyl)diacetate and dimethyl-2,2′-((5-formyl-2-methoxyphenyl)azanediyl)diacetate

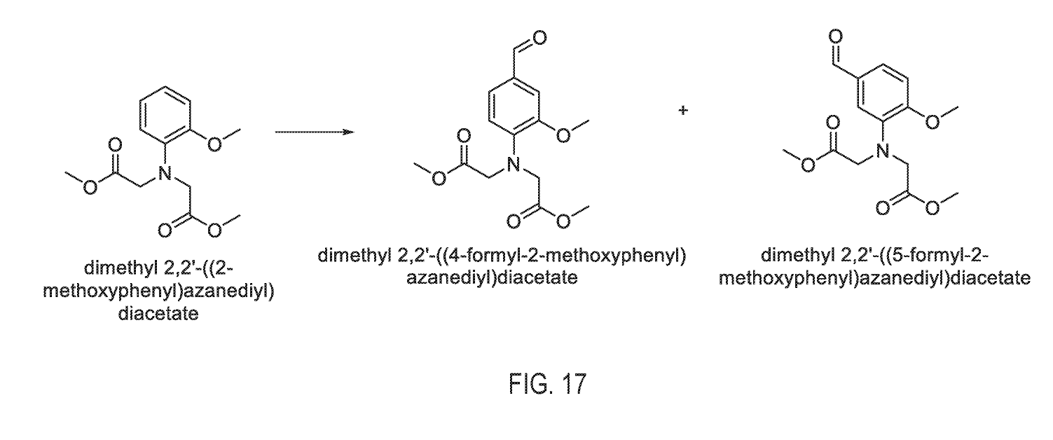

The reaction scheme for the synthesis of dimethyl-2,2′-((4-formyl-2-methoxyphenyl)azanediyl)diacetate and dimethyl-2,2′-5-formyl-2-methoxyphenyl)azanediyl is shown in FIG. 17. Phosphorus oxychloride (0.227 mL) was added drop wise to 2.0 ml, of DMF with ice/water cooling. The mixture was taken out of the cooling bath and stirred at RT for 20 min. Dimethyl 2,2′-((2-methoxyphenyl)azanediyl)diacetate (0.500 g; 1.87 mmol) was dissolved in 2 mL of DMF and added to the prepared Vilsmayer reagent. The flask was transferred into pre-heated 90° C. oil bath, stirred overnight with condenser under Ar atmosphere. The reaction mixture was diluted with sat. NaHCO₃ (30.0 mL) and the product was extracted with EtOAc (4×40.0 mL). The combined extract was washed with water (3×30.0 mL), brine (30.0 mL) and dried over Na₂SO₄. The solution was concentrated in vacuum, and the crude material was purified on silica gel column with EtOAc—hexane gradient (o-33%). Dimethyl 2,2′-((5-formyl-2-methoxyphenyl)azanediyl)diacetate (12.0 mg, 2%) eluted from the column first. The major product awed was dimethyl 2,2′-((4-formyl-2-methoxyphenyl)azanediyl)diacetate (370 mg, 67%).

### Example 7

Synthesis of N-(9-(3-(bis(2-(acetoxymethoxy)-2-oxoethyl)amino)-4-methoxyphenyl)-6-dimethylamino)-3H-xanthen-3-ylidene)-N-methylmethanaminium bromide

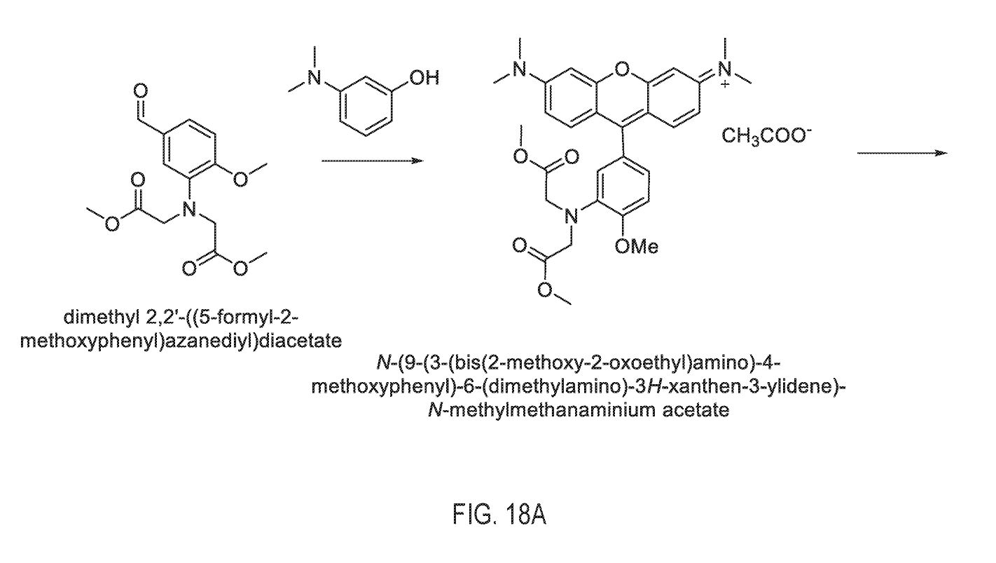

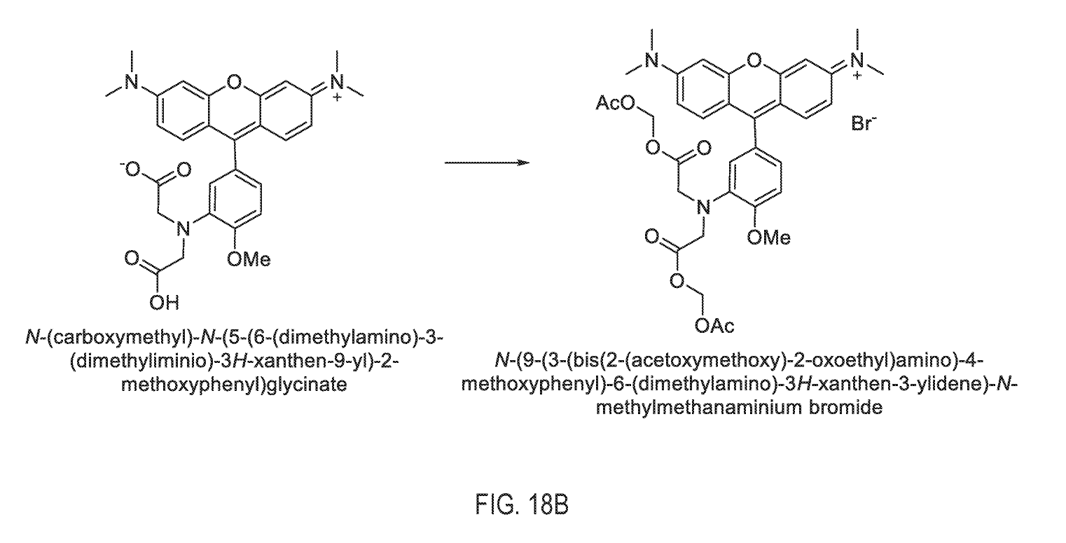

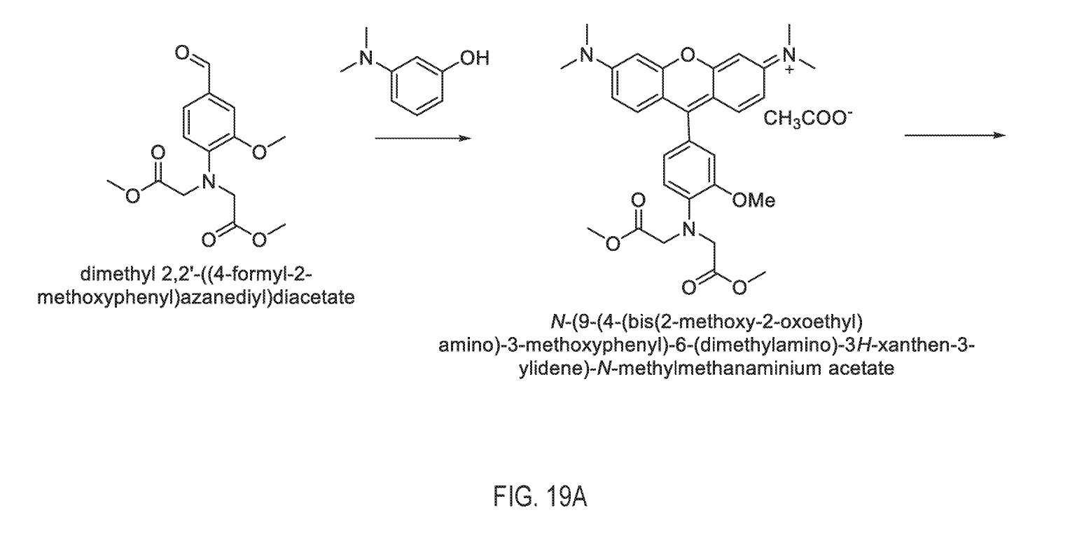

The reaction scheme for the synthesis of N-(9-(3-(bis(2-(acetoxymethoxy)-2-oxoethyl)amino)-4-methoxyphenyl)-6-(dimethylamino)-3H-xanthen-3-ylidene)-N-methylmethanaminium bromide is shown in FIG. 18A and FIG. 18B. 3-Dimethylaminophenol (0.11 g, 0.80 mmol) and dimethyl 2,2′-((5-formyl-2-methoxyphenyl)azanediyl)diacetate (0.11 g; 0.36 mmol) were dissolved in 1.0 mL of propionic acid; 1.0 mg of pTsOH was added to this solution, the reaction mixture was stirred under Ar atmosphere at 60° C. for 18 hr. The reaction mixture was removed from oil bath, and the most of propionic acid was removed on a rotary evaporator at 70° C. The residue was re-dissolved in 20 mL of chloroform and 20 mL of MeOH. Chloranil (0.26 g, 1.1 mmol) was added and mixture was stirred for ˜2 hrs at RT. The reaction mixture was concentrated to dryness and the crude material was purified on silica gel column with MeOH—chloroform gradient, containing 1.5% of AcOH (0-15% MeOH) to obtain N-(9-(3-(bis(2-methoxy-2-oxoethyl)amino)-4-methoxyphenyl)-6-(dimethylamino)-3H-xanthen-3-ylidene)-N-methylmethanaminium acetate (107 mg, 50%). N-(9-(3-(bis(2-methoxy-2-oxoethyl)amino)-4-methoxyphenyl)-6-(dimethylamion)-3H-xanthen-3-ylidene)-N-methylmethanaminium acetate (165 mg; 0.278 mmol) was dissolved in 10 mL of MeOH and 10 mL of dioxane; 1M potassium hydroxide (5 mL; 5 mmol) was added, and the resulting solution was stirred for 4 h at RT. Acetic acid (5.0 mL) was added to the mixture, and the solution was concentrated in vacuum. The residue was co-evaporated with MeOH/toluene to remove acetic acid. The crude product was purified on Biotage C18 120 g SNAP column with MeOH—25 mM TEAR buffer gradient (0-70%) to obtain N-(carboxymethyl)-N-(5-(6-(dimethylamino)-3-(diemthylimino)-3H-xanthen-9-yl)-2-methoxyphenyl)glycinate (83 mg, 50%). N-(carboxymethyl)-N-(5-(6-(dimethylamino)-3-(dimethyliminio)-3H-xanthen-9-yl)-2-methoxyphenyl)glycinate (16 mg; 0.026 mmol) was suspended in 1 mL of dry DMF. N,N,N-diisoptopylethylamine (20 μL; 0.115 mmol) was added to suspension with stirring, followed by adding bromomethyl acetate (14 μL; 0.14 mmol) and the mixture was stirred for 3 hrs at RT. The resulting solution was evaporated to dryness at 40° C. The crude product was re-dissolved in ACN (1.0 mL), and the solution added to 10 mL of ether. The suspension was centrifuged, supernatant discarded. The precipitate was re-dissolved in CHCl₃ and purified on small silica gel column with 5:1 DCM-MeOH mixture to obtain N-(9-(3-(bis(2-(acetoxymethoxy)-2-oxoethyl)amino)-4-methoxyphenyl)-6-(dimethylamino)-3H-xanthen-3- ylidene)-N-methylmethanaminium bromide (14 g, 73%).

### Example 8

### Synthesis of N-(9-(4-(bis(2-(acetoxymethoxy)-2-oxoethyl)amino)-3-methoxyphenyl)-6-(dimethylamino)-3H-xanthen-3-ylidene)-N-methylmethanaminium bromide

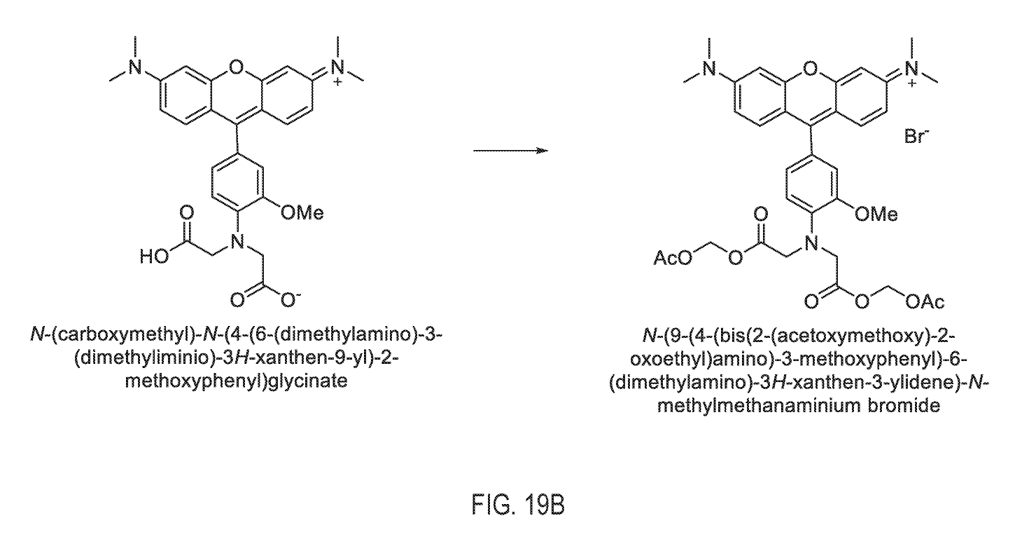

The reaction scheme for the synthesis of N-(9-(4-(bis(2-(acetoxymethoxy)-2-oxoethyl)amino)-3-methoxyphenyl)-6-(dimethylamino)-3H-xanthen-3-ylidene)-N-methylmethanaminium bromide is shown in FIG. 19A and FIG. 19B. 3-Dimethylaminophenol (0.54 g, 4.0 mmol) and dimethyl 2,2′-((4-formyl-2-methoxyphenyl)azanediyl)diacetate (0.53 g; 1.8 dissolved in 3 mL of propionic acid; 5.0 mg of pTsOH was added to this solution, the reaction mixture was stirred under Ar atmosphere at 60° C. for 18 hr. The reaction mixture was removed from oil bath, and the most of propionic acid was removed on rotary evaporator at 70° C. The residue was re-dissolved in 70 mL, of chloroform and 70 mL of MeOH, chloranil (1.3 g, 5.4 mmol) was added and mixture was stirred for ˜2 hrs at RT. The reaction mixture was concentrated to dryness and the crude material was purified on silica gel column with McOH—chloroform gradient, containing 1.5% of AcOH (0-15% MeOH) to obtain N-(9-(4-(bis(2-methoxy-2-oxoethyl)amino)-3-methoxyphenyl)-6-(dimethylamino)-3H-xanthen-3-ylidene)-N-methylmethanaminium acetate (823 mg, 77%). N-(9-(4-(bis(2-methoxy-2-oxoethyl)amino)-3-methoxyphenyl)-6-(dimethylamino)-3H-xanthen-3-ylidene)-N-methylmethanaminium acetate (823 mg; 1.39 mmol) was dissolved in 50 mL of MeOH and 50 mL of dioxane; 1M potassium hydroxide (25 mL; 25 mmol) was added, and the resulting solution was stirred for 4 hrs at RT. Acetic acid (5 mL) was added to the mixture, and the solution was concentrated in vacuum. The residue was co-evaporated with MeOH/toluene to remove acetic acid. The crude product was purified on Biotage C18 120 g SNAP colutrm with MeOH—25 mM TEAA buffer gradient (0-70%) to obtain N-(carboxymethyl)-N-(4-(6-(dimethylamino)-3-(dimethyliminio)-3H-xanthen-9-yl)-2-methoxyphenyl)glycinate (400 mg, 48%). N-(carboxymethyl-N-(4-(6-(dimethylamino)-3-(dimethyliminio)-3H-xanthen-9-yl)-2-methoxyphenyl)glycinate (80 mg; 0.13 mmol) was suspended in 3 mL of dry DMF. N,N,N-diisopropylethylamine (100 μL; 0.574 mmol) was added to suspension with stirring, followed by adding bromomethyl acetate (68 μL; 0.69 mmol) and the mixture was stirred for 3 hrs at RT. The resulting solution was evaporated to dryness at 40° C. The crude product was re-dissolved in ACN (1 ML), and the solution added to 10 mL of ether. The suspension was centrifuged, and the supernatant was discarded. The precipitate was re-dissolved in CHCl₃ and purified on small silica gel column with 5:1 DCM-MeOH mixture to obtain N-(9-(4-(bis(2-(acetoxymethoxy)-2-oxoethyl)amino)-3-methoxyphenyl)-6-(dimethylamino)-3H- xanthen-3-ylidene)-N-methylmethanaminium bromide (70 mg, 73%) (Compound 1) (see, FIG. 2).

### Example 9

### Synthesis of N-(10-(4-(bis(2-(acetoxymethoxy)-2-oxoethyl)amino)-3-methoxyphenyl)-7-(dimethylamino)-9,9-dimethylanthracen-2(9H)-yliden-N-methylmethanaminium trifluoromethanesulfonate and N-(10-(3-(bis(2-(acetoxymethoxy)-2-oxoethyl)amino)-4-methoxyphenyl)-7-(dimethylamino)-9,9-diemthylanthracen-2(9H)-ylidene)-N-methylmethanaminium trifluoromethanesulfonate

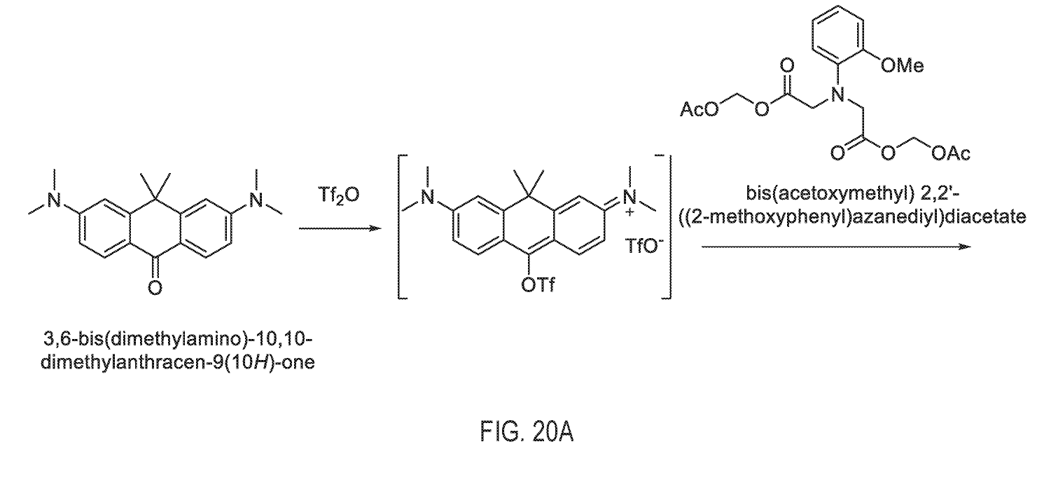

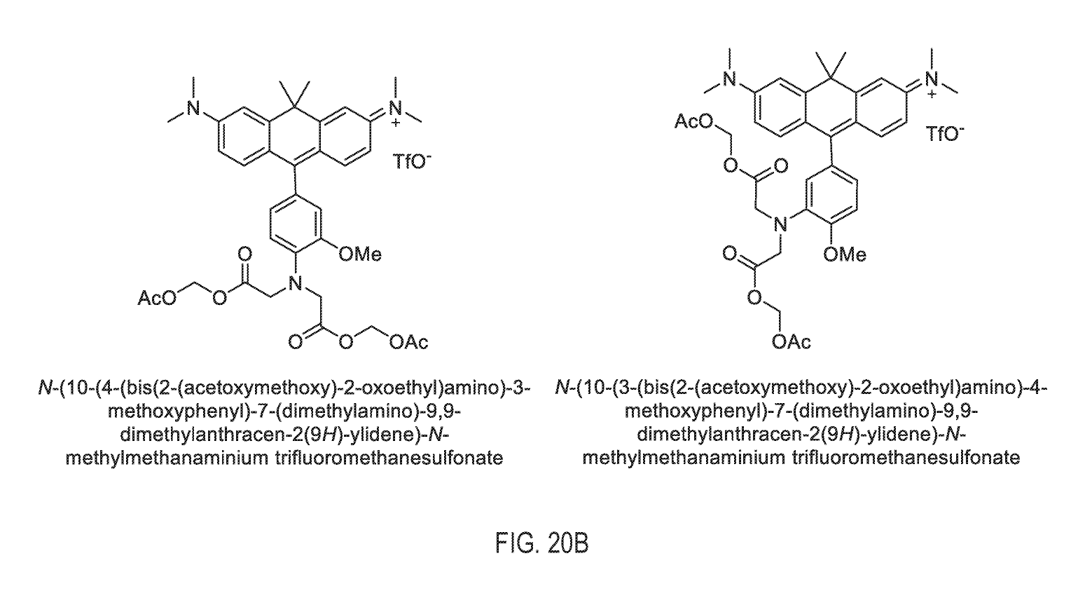

The reaction scheme for the synthesis of N-(10-(4-(bis(2-(acetoxymethoxy)-2-oxoethyl)amino)-3-methoxyphenyl)-7-(dimethylamino)-9,9-dimethylanthracen-2(9H)-ylidene)-N- methylthanaminium rifluoromethanesulfonate and N-(10-(3-(bis-(acetoxymethoxy)-2-oxoethyl)amino)-4-methoxyphenyl)-7-(dimethylamino)-9,9-dimethylanthracen-2(9H)-ylidene)-N-methylmethanaminium trifluoromethanesulfonate is shown in FIG. 20A and FIG. 20B. 3,6-bis(dimethylamino)-10,10-dimethylanthracen-9(10H)-one (300 mg, 0.973 mmol) was dissolved in 10 mL of dry DCM and solution was cooled ice/water bath. Triflic anhydride (330 mg, 1.17 mmol) was added to the solution and reaction mixture was stirred for 1 hr in the cooling bath. Bis(acetoxymethyl) 2,2′-((2-methoxyphen azanediyl)diacetate was added to the above solution and the reaction mixture was allowed to warm to RT and stirred overnight. The resulting solution was diluted with 20 mL of DCM and extracted with sat. NaHCO₃ solution (20 mL), brine (20 mL), dried over Na₂SO₄ and evaporated. The crude mixture of products was separated on silica gel column with EtOAc—chloroform gradient (0-60%) to obtain N-(10-(4-(bis(2-(acetoxymethoxy)-2-oxoethyl)amino)-3-methoxyphenyl-7-(dimethylamino)-9,9-dimethylanthracen-2(9H)-ylidene)-N-methylmethanaminium trifluoromethanesulfonate (80 mg, 10%) and N-(10-(3-(bis(2-(acetoxymethoxy)-2-oxoethyl)amino)-4-methoxyphenyl)-7-(dimethylamino)-9,9-dimethylanthracen-2(9H)-ylidene)-N-methylmethanaminium trifluoromethanesulfonate (20 mg, 2%).

### Example 10

### Thallium Ion Influx Assay

This example describes components and methods that can be used to detect thallium ion influx through an ion channel.

Component A: Thallium ion indicator dye in amount that will prepare 1-10 uM solution in normal Chloride Assay Buffer.
Component B: Physiological (normal) Chloride Assay Buffer: NaCl 145 mM; KCl 2.5 mM; CaCl₂ 1.8 mM; MgCl₂ 1.0 mM; HEPES 20 mM; pH set to 7.4 with NaOH.
Component C: Chloride Free Stimulus Buffer: 140 mM Na Gluconate; 2.5 mM K Gluconate; 1.8 m141 Ca Gluconate; 1.0 mM Mg Gluconate; 10 mM HEPES; 25 mM K₂SO₄; 5.0 mM Tl₂SO₄.

Cells including an ion channel are cultured to about 75% confluence and in log-phase growth. The cells are then harvested using well-known cell culture techniques and buffers and replated into a 96 well plate at a density of, for example, 5,000-40,000 cells per well (e.g., 20,000 cells per well). The cells comprise the ion channel endogenously, or the cells can be engineered to comprise ion channels of a particular type. Methods of transfecting and/or transforming cells to express a particular type of protein are well known in the art and need not be repeated herein.

To prepare the dye loading buffer, Component A is prepared in assay buffer (Component B) at concentrations from 1-20 micromolar. Optional addition of 1-100 μg/mL Pluronic surfactant can aid-in dispersion and loading of dye and/or 1-10 mM probenecid to aid in retention of the dye. Next the cell culture media is aspirated from the cells in the 96 well plate and replaced with dye loading buffer and incubated at room temperature for 60-120 minutes. While the cells are incubating in loading buffer, stimulus buffer (Component C) is prepared for addition to the microplate. The loading buffer then is removed from the cells, and replaced with Component A that contains no dye, to reduce background fluorescence from unincorporated dye in the solution. Next, the fluorescence from the cells is read on a spectrophotometer. For samples implementing a thallium indicator that emits in the green spectral region, the sample is excited with light in the range of 470-500 nm, and emission is read at 520 to 540 nm, with a filter cutoff of 515 nm. For samples implementing a thallium indicator that emits in the red spectral region, the sample is excited with light in the range of 545-565 nm and read at 580-600 nm, with a filter cutoff of 570 nm. A pre-stimulus “baseline” signal is measured at regular intervals for 10-30 seconds in advance of adding Stimulus Buffer (Component C). Component C is added to the cells in a 1:5 dilution at the indicated time, and the increase in fluorescence from the cells is measured over time.

### Example 11

### Assay of Cells Transfected with hERG Ion Channels

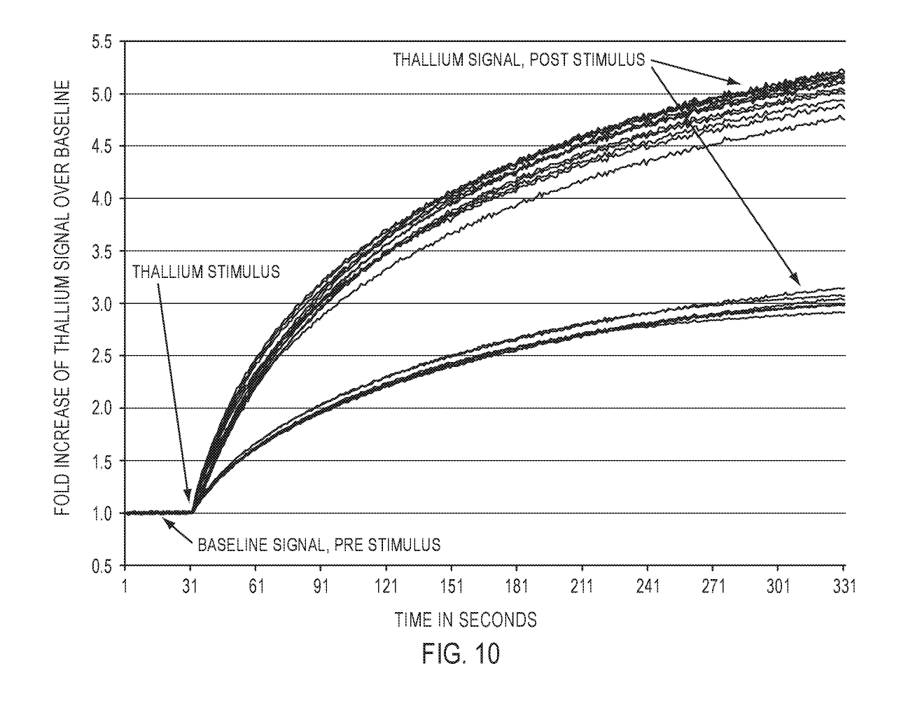

Cells expressing the hERG potassium ion channel were assayed using the method described in Example 10 to compare the efficacy of two different isomeric thallium ion indicators. CHO cells expressing the hERG ion channel were assayed using methods described herein. Cells were loaded in assay buffer containing the indicated concentration of dye in Table 1 (i.e., 1 or 10 μM) washed with dye-free assay buffer (Component B) and the stimulus buffer (Component C) was delivered to the cells in a volume of 25 μL, added to 100 μL for a 5× dilution (Component C, containing 25 mM T K₂SO₄ and 10 mM Tl₂SO₄). In this particular example, the final concentration of potassium on the cells was 10 mM and the final concentration of thallium on the cells was 2 mM. FIG. 10 shows the fluorescence curves of cells (plotted as dF/F as a function of time) expressing the hERG ion channel and assayed using the methods described herein from cells loaded with Compound 9 (lower races) or Compound 8 (upper traces). Fluorescence data from the samples was plotted over time as fold increase in signal (post stimulus) over baseline (pre stimulus). Signal amplitude was compared from an average of 5-10 individual wells loaded with the dye indicated. Both compounds detected the presence of thallium ions. However, the larger response from Compound 8 indicated that it was far more sensitive to thallium ions than Compound 9 in the assay.

### Example 13

### Comparison of Different Thallium Sensitive Compounds in the Thallium Influx Assay

Cells expressing the hERG potassium ion channel were assayed using the thallium influx method described in Example 11 to compare the efficacy of various thallium ion indicators disclosed herein. Thallium indicators were prepared in loading buffer at the concentrations listed in Table 1 and loaded into cells for 60 minutes. The samples were excited with light in the range of 470-500 nm, and emission was read at 520 to 540 nm with a filter cutoff of 515 nm, with the exception that Compound 1 was excited with light in the range of 545-565 nm and read at 580-600 nm, with a filter cutoff of 570 nm. The fold increase of signal over baseline for the tested compounds is summarized in Table 1.

Referring to Table 1, the fluorogenic response for the non-fluorinated analogues was particularly sensitive to changes in dye concentration. Surprisingly, the sensitivity to dye concentration depended on which structural isomer was tested. In particular, the para isomer for a parent structure exhibited a higher fold increase of fluorescence signal over baseline relative to that measured for the meta isomer at the same concentration of dye loading. For example, the fold increase for the para isomer of a non-fluorinated analogue (e.g., Compound 5) decreased significantly when the dye concentration was increased frons 1 μM to 2 μM, while the meta isomer of the non-fluorinated analogue (e.g., Compound 2) experienced little to no change in fold under the same assay conditions. In contrast, the fluorogenic response for the fluorinated para (e.g., Compound 8) and meta isomer (Compound 9) of the same parent compound was not affected by the 1 μM increase in dye loading concentration. Because the fluorinated analogues can be used over a wider range of dye concentrations than their non-fluorinated counterparts, regardless of isomeric form, these compounds can serve as particularly robust thallium indicators in the assays described herein.

TABLE 1
Fluorogenic Responses for Thallium Ion Indicators

| Compound | Concentration (μM) | Fold Increase Over Baseline |
|---|---|---|
| 1 | 10 | 2.7 |
| 2 | 1 | 1.2 |
| 2 | 2 | 1.3 |
| 3 | 1 | 1.1 |
| 3 | 2 | .08 |
| 4 | 1 | 1.5 |
| 4 | 2 | 1.4 |
| 5 | 1 | 3.7 |
| 5 | 2 | 3.1 |
| 6 | 1 | 3.7 |
| 6 | 2 | 2.8 |
| 7 | 1 | 2.3 |
| 7 | 2 | 1.5 |
| 8 | 1 | 3.4 |
| 8 | 2 | 3.4 |
| 9 | 1 | 2.0 |
| 9 | 2 | 2.0 |

### Example 14

### Thallium Ion Sensitivity Assay

This example describes an assay to evaluate a compound's sensitivity to thallium (I) ions. Compounds with sufficient sensitivity to thallium (I) ions can be used as thallium ion indicators for measuring thallium ion influx and efflux through ion channels. To characterize the best response for a compound, a loading buffer solution is prepared, as described, containing the dye at concentrations between 1 and 20 micromolar and the fluorescence response from the cells was measured in response to a thallium ion stimulus. Dyes with larger signal increases over baseline are considered superior in the assay.

All of the U.S. patents, U.S. patent application publications, U.S. patent applications, foreign patents, foreign patent applications and non-patent publications referred to M this specification are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not he construed to limit the claims to the specific embodiments disclosed in the specification and the claims, but should be construed to include all possible embodiments along with the full scope of equivalents to which such claims are entitled. Accordingly, the claims are not limited the disclosure.

1. A method for detecting the activity of a potassium ion channel in a cell, comprising:
a) contacting the cell with a loading buffer, wherein the cell comprises a potassium ion channel, wherein the loading buffer comprises a thallium ion indicator;
b) applying a stimulus buffer to the cell, wherein the stimulus buffer comprises thallium ions, thereby causing thallium ion influx into the cell through the potassium ion channel; and
c) measuring a change in at least one optical property of the thallium ion indicator in response to thallium influx, thereby detecting the activity of the potassium ion channel,
wherein the thallium ion indicator has a structure represented as: 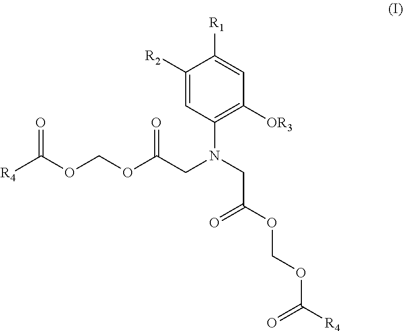
wherein R₂=H and R₁= 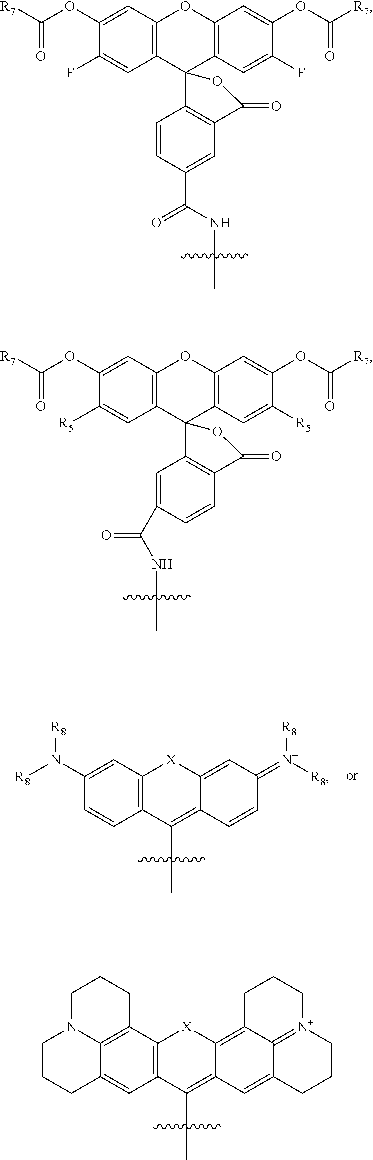
or wherein R₁=H and R₂= 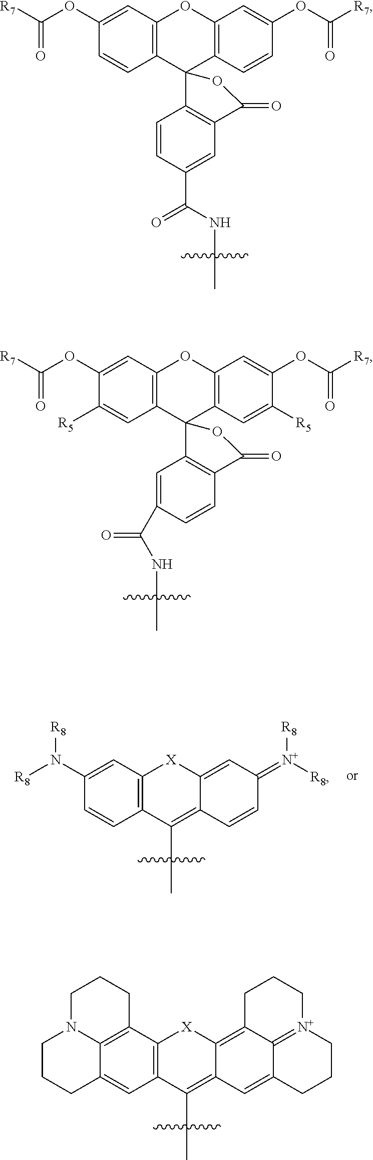
wherein X=O or (R₆)₂C;
wherein R₃, R₄, R₆ and R₈ are independently C₁-C₆ alkyl;
wherein R₅ is H or F; and
wherein R₇ is H, CH₃ or C₂-C₆ alkyl, or a salt thereof.

2. The method claim 1, wherein the stimulus buffer comprises thallium ion concentrations of less than about 4.5 mM.

3. The method of claim 1, further comprising quantifying the level of thallium ion influx.

4. The method of claim 1, wherein the at least one optical property of the thallium indicator is intensity, polarity, frequency, or optical density.

5. The method of claim 1, wherein the at least one optical property is luminescence intensity, wherein the method further comprises measuring a change in the luminescence intensity of the thallium ion indicator in response to thallium ion influx.

6. The method of claim 1, wherein the loading buffer is chloride free.

7. The method of claim 1, wherein the loading buffer further comprises a physiological concentration of chloride ions.

8. The method of claim 1, wherein the cell is a mammalian cell.

9. (canceled)

10. The method of claim 1, wherein the thallium ions are in the form of a salt.

11. The method of claim 10, wherein the thallium salt is soluble in the loading buffer.

12. The method of claim 10, wherein the thallium salt is Tl₂SO₄, Tl₂CO₃, TlCl, TlOH, TlOAc, or TlNO₃.

13. The method of claim 1, further comprising adding an extracellular quencher to the loading buffer solution, whereby the emission of extracellular thallium ion indicator is quenched.

14. (canceled)

15. (canceled)

16. (canceled)

17. A compound having a structure represented as: 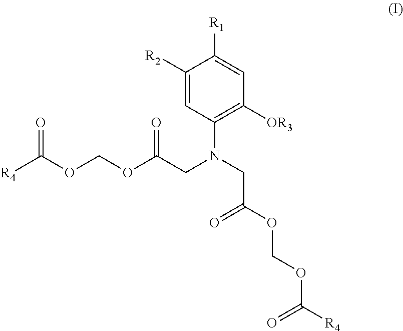
wherein R₂=H and R₁= 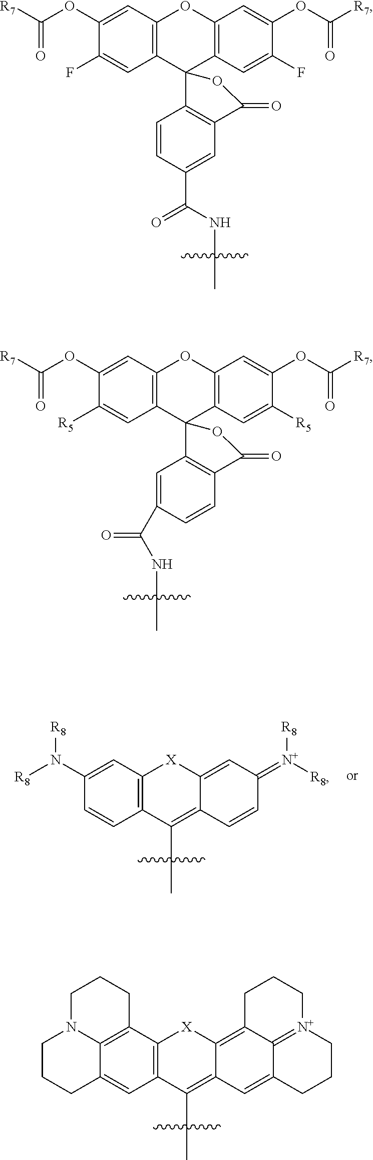
wherein X=O or (R₆)₂C;
wherein R₃, R₄, R₆ and R₈ are independently C₁-C₆ alkyl;
wherein R₅ is H or F; and
wherein R₇ is H, CH₃ or C₂-C₆ alkyl, or a salt thereof, with the exception that if R₁ is 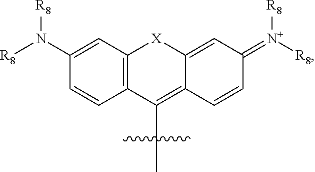
then each R₈ is not C₁ alkyl.

18. A compound having a structure represented as: 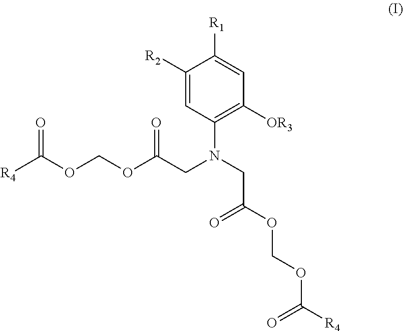
wherein R₁=H and R₂= 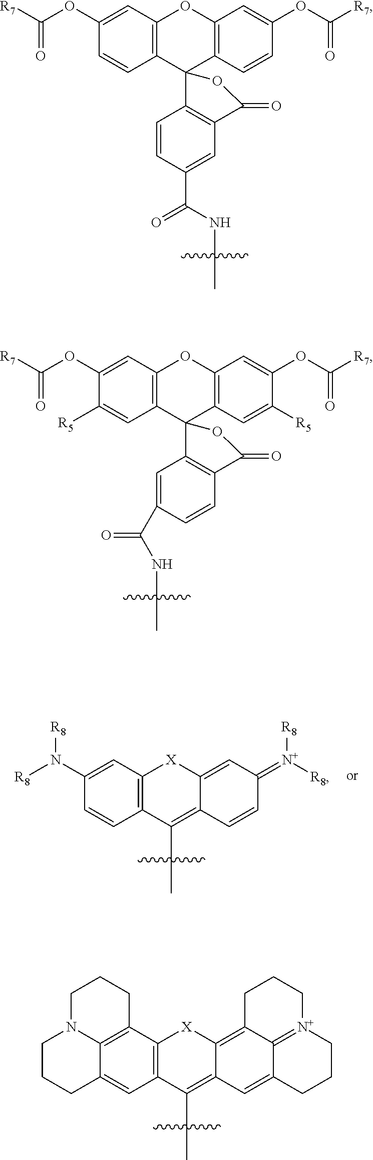
wherein X=O or (R₆)₂C;
wherein R₃, R₄, R₆ and R₈ are independently C₁-C₆ alkyl;
wherein R₅ is H or F; and
wherein R₇ is H, CH₃ or C₂-C₆ alkyl, or a salt thereof.

19. The compound of claim 17 or 18, wherein at least one R₅, if present, is F.

20. The method of claim 1, wherein at least one R₅, if present, is F.

21. (canceled)

22. A fluorescent complex comprising:
a compound of claim 17 or 18; and
a thallium ion, wherein the complex emits light upon excitation at an appropriate spectral wavelength.

23. (canceled)

24. (canceled)

25. A kit for detecting the activity of a potassium ion channel in a cell, the kit comprising:
a loading buffer solution, wherein the loading buffer solution, a thallium ion indicator according to claim 17 or claim 18; and a stimulus buffer, wherein the stimulus buffer comprises thallium ions, and wherein the stimulus buffer causes thallium ion influx into the cell through the ion channel.
